SEQUENCE LISTING (14 sequences)

SEQ ID NO: 1 - DNA, 546 bp, Homo sapiens
  CDS (1)..(543)
ggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctga

SEQ ID NO: 2 - protein, 181 aa, Homo sapiens
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 3 - DNA, 489 bp, Homo sapiens
  CDS (1)..(489)
cgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttctattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgtctctgcgtacctctacccatccggaatctacc

SEQ ID NO: 4 - protein, 163 aa, Homo sapiens
RFKSLSPQELASFKKARDALEESLKLKNWSCSSPVFPGNWDLRLLQVRERPVALEAELALTLKVLEAAAGPALEDVLDQPLHTLHHILSQLQASIQPQPTAGPRPRGRLHHWLHRLQEAPKKESAGCLEASVTFNLFRLLTRDLKYVADGNLSLRTSTHPEST

SEQ ID NO: 5 - DNA, 483 bp, Homo sapiens
  CDS (1)..(483)
aaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgat

SEQ ID NO: 6 - protein, 161 aa, Homo sapiens
KPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVAD

SEQ ID NO: 7 - DNA, 471 bp, Homo sapiens
  CDS (1)..(471)
aaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaa

SEQ ID NO: 8 - protein, 157 aa, Homo sapiens
KPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLK

SEQ ID NO: 9 - DNA, 447 bp, Homo sapiens
  CDS (1)..(447)
cgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttctattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgat

SEQ ID NO: 10 - protein, 149 aa, Homo sapiens
RFKSLSPQELASFKKARDALEESLKLKNWSCSSPVFPGNWDLRLLQVRERPVALEAELALTLKVLEAAAGPALEDVLDQPLHTLHHILSQLQASIQPQPTAGPRPRGRLHHWLHRLQEAPKKESAGCLEASVTFNLFRLLTRDLKYVAD

SEQ ID NO: 11 - DNA, 435 bp, Homo sapiens
  CDS (1)..(435)
cgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttctattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaa

SEQ ID NO: 12 - protein, 145 aa, Homo sapiens
RFKSLSPQELASFKKARDALEESLKLKNWSCSSPVFPGNWDLRLLQVRERPVALEAELALTLKVLEAAAGPALEDVLDQPLHTLHHILSQLQASIQPQPTAGPRPRGRLHHWLHRLQEAPKKESAGCLEASVTFNLFRLLTRDLK

SEQ ID NO: 13 - DNA, 531 bp, Homo sapiens
  CDS (1)..(531)
atgaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgtctctgcgtacctctacccatccggaatctacctaa

SEQ ID NO: 14 - protein, 176 aa, Homo sapiens
MKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLSLRTSTHPEST

(19) United States
(12) Patent Application Publication
(10) Pub. No.: US 2011/0243888 A1
(43) Pub. Date: Oct. 6, 2011

(54) IL-29 MUTANTS AND USES THEREOF

(76) Inventors: Paul O. Sheppard, Granite Falls, WA (US); Henrik Andersen, Seattle, WA (US)

(21) Appl. No.: 13/129,042

(22) PCT Filed: Nov. 20, 2009
(86) PCT No.: PCT/US09/65398

§ 371 Date: Jun. 21, 2011

(30) Foreign Application Priority Data
Nov. 20, 2008 (US) 61/116589

(51) Int. Cl. A61K 38/20; C07K 14/54; C08G 65/48; C07H 21/04; C12N 15/63; C12N 5/10; C12N 1/00; C12P 21/02; C07K 16/24; A61P 31/14; A61P 31/20

(52) U.S. Cl. 424/85.2; 530/351; 525/54.1; 536/23.5; 435/320.1; 435/325; 435/243; 435/419; 435/69.52; 530/387.9; 530/387.3

(57) ABSTRACT

The present invention relates to truncated IL-29 mutant molecules and methods of using same. The truncated IL-29 molecules can be used to treat viral infections, such as hepatitis C, autoimmune disorders and various types of cancer.

### BACKGROUND OF THE INVENTION

It has been estimated that 3% of the world's population, i.e., 130 million individuals are infected with hepatitis C. Stauber R E and Stadlbauer V., Journal of Clinical Virology, 36:87-94 (2006). The majority have been infected via parenteral exposure with contaminated injections, either related to injection drug use or contaminated injections or transfusion with blood products received as part of an individual' health care. The current standard of care for hepatitis C is pegylated interferon (PEG-IFN) alpha (given once weekly) in combination with oral ribavirin (given daily). Heathcote J. and Main J., Journal of Viral Hepatitis, 12:223-235 (2005).

Chronic infection with hepatitis C virus (HCV) is a leading cause of cirrhosis, liver failure, and hepatocellular carcinoma in the United States and worldwide. The primary goal of treatment is to eradicate the virus and prevent development of long-term complications. Successful treatment is defined as achievement of a sustained virologic response (SVR) as evidenced by undetectable HCV RNA levels at least 6 months following discontinuation of therapy (Pearlman B L. Hepatitis C treatment update. Am J Med 2004; 117(5):344-352).

For patients infected with genotype 1 HCV, the most common genotype in the United States, treatment consists of weekly administration of a PEGylated interferon alpha (PEG-IFN-α) in combination with daily ribavirin for 48 weeks. The two currently approved forms of PEG IFN-α are peginterferon alpha 2a (PEGASYS®), and peginterferon alpha-2b (PEG-INTRON®), both of which are associated with SVR rates of approximately 50% in patients infected with genotype 1 HCV (Seeff L B. Natural history of chronic hepatitis C. Hepatology 2002A; 36(5 Suppl 1):535-46; Strader D B, Wright T, Thomas D L, Seeff L B. Diagnosis, management, and treatment of hepatitis C. Hepatology 2004; 39(4):1147-1171). For those patients who fail to achieve an SVR, there is currently no standard treatment.

Relapsed patients, who compose approximately 20% of all treated genotype 1 HCV patients, represent a unique population of PEG-IFN-α treatment failures (Hadziyannis S J, Sette H, Jr., Morgan T R, Balan V, Diago M, Marcellin P, Ramadori G, Bodenheimer H, Jr., Bernstein D, Rizzetto M, Zeuzem S, Pockros P J, Lin A, Ackrill A M. Peginterferon-alpha2a and ribavirin combination therapy in chronic hepatitis C: a randomized study of treatment duration and ribavirin dose. Ann Intern Med 2004; 140(5):346-355). While these patients have undetectable HCV RNA levels at the end of treatment, they relapse with detectable HCV RNA levels less than 6 months later (Hoothagle J H, Seeff L B. Peginterferon and ribavirin for chronic hepatitis C. N Engl J Med 2006; 355(23):2444-2451). Factors contributing to relapse may include dose reduction in ribavirin, especially during the first 24 weeks of treatment (Shiffman M L. Chronic hepatitis C: treatment of pegylated interferon/ribavirin nonresponders. Curr Gastroenterol Rep 2006; 8(1):46-52.). Upon retreatment with IFN α based therapy, relapsed patients may manifest decreases in HCV RNA levels similar to those seen during their prior course of therapy (Strader D B, Wright T, Thomas D L, Seeff L B. Diagnosis, management, and treatment of hepatitis C. Hepatology 2004; 39(4):1147-1171), and in cases where prior therapy consisted of a non-PEGylated IFN-α, may be able to achieve an SVR with retreatment utilizing a PEG-IFN-α and ribavirin (Jacobson I M, et al., A randomized trial of pegylated interferon alpha-2b plus ribavirin in the retreatment of chronic hepatitis C. Am J Gastroenterol 2005; 100(11):2453-2462; Mathew A, et al., Sustained viral response to pegylated interferon alpha-2b and ribavirin in chronic hepatitis C refractory to prior treatment. Dig Dis Sci 2006; 51(11):1956-1961; Shiffman M L., Chronic hepatitis C: treatment of pegylated interferon/ribavirin nonresponders. Curr Gastroenterol Rep 2006; 8(1):46-52). This pattern of failure and response to retreatment suggests that relapsed patients retain the potential to respond to interferon-based therapy and therefore are a unique population in which to study the potential effects of novel interferon-like molecules (Hoofnagle J H, Seeff L B. Peginterferon and ribavirin for chronic hepatitis C. N Engl J Med 2006; 355(23):2444-2451; FDA CDER Antiviral Drugs Advisory Committee. Summary Minutes of the Antiviral Drugs Advisory Committee, Oct. 19-20, 2006).

Treatment with PEG-IFN-α and ribavirin is associated with significant side effects. Major toxicities of PEG-IFN-α include flu-like symptoms; hematologic abnormalities including neutropenia, thrombocytopenia, and anemia; and neuropsychiatric disorders, most commonly depression. Other toxicities include gastrointestinal disturbances and dermatologic, autoimmune, and cardiac conditions. Elevations in liver transaminases have also been reported, particularly with peginterferon alpha 2a (Gish R G. Treating hepatitis C: the state of the art. Gastroenterol Clin North Am 2004; 33(1 Suppl):S1-9; Hoffmann-La Roche Inc. Package Insert: PEGASYS(R) (peginterferon alfa-2a). 2005B:1-46). Ribavirin is associated with a number of adverse effects, most notably hemolytic anemia, which in combination with the myelosuppressive effects of IFN-α can be a significant clinical problem (Kowdley K V. Hematologic side effects of interferon and ribavirin therapy. J Clin Gastroenterol 2005; 39(1 Suppl):S3-8; Strader D B, Wright T, Thomas D L, Seeff L B. Diagnosis, management, and treatment of hepatitis C. Hepatology 2004; 39(4):1147-1171).

The toxicities associated with PEG-IFN-α and ribavirin often lead to delays in starting therapy, as well as dose reductions and early discontinuation of treatment (Pearlman B L. Hepatitis C treatment update. Am J Med 2004; 117(5):344-352), all of which decrease the likelihood of achieving SVR. Adherence to therapy (defined as receiving ≧80% of the prescribed PEG IFN-α dose and ≧80% of the ribavirin dose for the duration of therapy) has been associated with higher SVR rates in genotype 1 HCV patients (McHutchison J G, et al., Adherence to combination therapy enhances sustained response in genotype-1-infected patients with chronic hepatitis C. Gastroenterology 2002; 123(4):1061-1069).

Given the limitations of current therapy, there remains a need for improved treatments for HCV and other diseaeses. Thus, there remains a need in the art for Type III Interferons that can be developed into potent therapeutics, while being effectively and efficiently produced at large-scale in any number of available production systems. The truncated Type III Interferon polypeptides of the present invention are such a therapeutic.

### DETAILED DESCRIPTION OF THE INVENTION

### Definitions

In the description that follows, a number of terms are used extensively. The following definitions are provided to facilitate understanding of the invention.

The terms “a,” “an,” and “the” include plural referents, unless the context clearly indicates otherwise.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

A “fusion protein” or a “fusion polypeptide” is a hybrid protein or polypeptide expressed by a nucleic acid molecule comprising nucleotide sequences of at least two genes of portions thereof. For example, a fusion protein can comprise at least part of a Fc domain fused with a Type III Interferon polypeptide.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The terms “nucleic acid” or “nucleic acid molecule” refer to a deoxyribonucleotide or ribonucleotide polymer in either single- or double-stranded form, and unless otherwise limited, would encompass known analogs of natural nucleotides that can function in a similar manner as naturally occurring nucleotides. A “nucleotide sequence” also refers to a polynucleotide molecule or oligonucleotide molecule in the form of a separate fragment or as a component of a larger nucleic acid. The nucleotide sequence or molecule may also be referred to as a “probe” or a “primer.” Some of the nucleic acid molecules of the invention are derived from DNA or RNA isolated at least once in substantially pure form and in a quantity or concentration enabling identification, manipulation, and recovery of its component nucleotide sequence by standard biochemical methods. Examples of such methods, including methods for PCR protocols that may be used herein, are disclosed in Sambrook et al., Molecular Cloning: A Laboratory Manual, 2nd ed., Cold Spring Harbor Laboratory Press, New York (1989), Ausubel, F. A., et al., eds., Current Protocols in Molecular Biology, John Wiley and Sons, Inc., New York (1987), and Innis, M., et al., (Eds.) PCR Protocols: A Guide to Methods and Applications, Academic Press, San Diego, Calif. (1990). Reference to a nucleic acid molecule also includes its complement as determined by the standard Watson-Crick base-pairing rules, with uracil (U) in RNA replacing thymine (T) in DNA, unless the complement is specifically excluded. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like.

As described herein, the nucleic acid molecules of the invention include DNA in both single-stranded and double-stranded form, as well as the DNA or RNA complement thereof. DNA includes, for example, DNA, genomic DNA, chemically synthesized DNA, DNA amplified by PCR, and combinations thereof. Genomic DNA, including translated, non-translated and control regions, may be isolated by conventional techniques, e.g., using any one of the cDNAs of the invention, or suitable fragments thereof, as a probe, to identify a piece of genomic DNA which can then be cloned using methods commonly known in the art.

A “nucleic acid molecule construct” is a nucleic acid molecule, either single-stranded or double-stranded, that has been modified through human intervention to contain segments of nucleic acid combined and juxtaposed in an arrangement not existing in nature.

The term “operably linked”, when referring to DNA segments, indicates that the segments are arranged so that they function in concert for their intended purposes, e.g., transcription initiates in the promoter and proceeds through the coding segment to the terminator.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

A “recombinant host” is a cell that contains a heterologous nucleic acid molecule, such as a cloning vector or expression vector. In the present context, an example of a recombinant host is a cell that produces a multispecific antibody or antibody fragment of the present invention from an expression vector.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

A “therapeutically effective amount” of a composition is that amount that produces a statistically significant effect, such as a statistically significant reduction in disease progression or a statistically significant improvement in organ function. The exact dose will be determined by the clinician according to accepted standards, taking into account the nature and severity of the condition to be treated, patient traits, etc. Determination of dose is within the level of ordinary skill in the art.

A polypeptide “variant” as referred to herein means a polypeptide substantially homologous to a native polypeptide, but which has an amino acid sequence different from that encoded by any of the nucleic acid sequences of the invention because of one or more deletions, insertions or substitutions. Amino acid sequence insertions include amino- and/or carboxyl-terminal fusions ranging in length from one residue to polypeptides containing a hundred or more residues, as well as intrasequence insertions of single or multiple amino acid residues. Intrasequence insertions (e.g., insertions within the target polypeptide sequence) may range generally from about 1 to 10 residues, more preferably 1 to 5, most preferably 1 to 3. Variants can comprise conservatively substituted sequences, meaning that a given amino acid residue is replaced by a residue having similar physiochemical characteristics. See, Zubay, Biochemistry, Addison-Wesley Pub. Co., (1983). It is a well-established principle of protein and peptide chemistry that certain amino acids substitutions, entitled “conservative” amino acid substitutions, can frequently be made in a protein or a peptide without altering either the confirmation or the function of the protein or peptide. Such changes include substituting any of isoleucine (I), valine (V), and leucine (L) for any other of these amino acids; aspartic acid (D) for glutamic acid (E) and vice versa; glutamine (Q) for asparagine (N) and vice versa; and serine (S) for threonine (T) and vice versa. Ordinarily, variants will have an amino acid sequence having at least 75% amino acid sequence identity with the reference sequence, more preferably at least 80%, more preferably at least 85%, more preferably at least 90%, and most preferably at least 95%. Identity or homology with respect to this sequence is defined herein as the percentage of amino acid residues in the candidate sequence that are identical with the reference sequence residues, after aligning the sequences and introducing gaps, if necessary, to achieve the maximum percent sequence identity. Preferably, variants will retain the primary function of the parent from which it they are derived.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

The interferon lambdas are a newly described family of cytokines, related to both type-1 interferons and IL-10 family members. The family, classified as the “Type III” Interferons, is comprised of three novel four helical bundle cytokines designated as IFN-λ1, IFN-λ2 and IFN-λ3 (also referred to as IL-29 or zcyto21, IL-28A or zcyto20, and IL-28B or zcyto22, respectively). Jordan W J et al., Genes and Immunity, 8:13-20 (2007). All three interferons lambdas signal through a heterodimeric receptor complex composed of the class II cytokine receptors IL-28RA (also known as IL-28 receptor alpha) and CRF2-4 (also known as IL-10RB or IL-10R2. The IL-28 receptor is quite distinct from that used by Type I Interferons.

IFN-λ1 or IL-29 is a member of the recently described Type III interferon family (Kotenko S V et al., IFN-lambdas mediate antiviral protection through a distinct class II cytokine receptor complex. Nat Immunol 2003; 4(1):69-77; Sheppard P et al., IL-28, IL-29 and their class II cytokine receptor IL-28R. Nat Immunol 2003; 4(1):63-68) with functional similarities to Type I interferons, which include IFN-α and IFN-β (Ank, et al., 2006). Similarly to IFN-α, the Type III interferons are induced in response to viral infection and stimulates an intracellular response that involves phosphorylation of signal transducing activator of transcription (STAT) proteins and induction of interferon-responsive genes, also known as interferon stimulated genes (ISGs). ISGs encode proteins involved in antiviral responses and immune stimulation, including Protein kinase R (PkR), Myxovirus resistance (Mx), 2′5′ oligoadenylate synthetase (OAS), and β2-microglobulin (B2M) (Samuel C E. Antiviral actions of interferons. Clin Microbiol Rev 2001; 14(4):778-809; Stark G R, Kerr I M, Williams B R, Silverman R H, Schreiber R D. How cells respond to interferons Annu Rev Biochem 1998; 67:227-264).

The present invention provides novel polynucleotide molecules, including DNA and RNA molecules, that encode mutants of IL-29.

Wild-type IL-29 gene (SEQ ID NO:1) encodes a polypeptide of 181 amino acids, as shown in SEQ ID NO:2. The IL-29 polypeptide base sequence of SEQ ID NO:2 and other IL-29 sequences have been previously disclosed in the prior art in, see for example, U.S. Pat. Nos. 7,038,032, 6,927,040, and 7,157,559 and U.S. Patent Publication No. 2007/0053933, all of which are herein incorporated by reference in their entirety. WO 07/041,713 discloses methods of manufacturing IL-29 polypeptides and is herein incorporated by reference in its entirety. Specifically incorporated by reference of WO 07/041,713 is its teaching of the expression, fermentation, recovery, solubilization of inclusion bodies, clarification and concentration of refolded IL-29, purification, pegylation and purification of pegylated IL-29.

The IL-29 polypeptides of the present invention were constructed in light of Met IL-29 d2/7 C5 mutant (SEQ ID NOs:13/14). The polypeptide sequence of SEQ ID NO:14 has an additional N-terminal methionine as compared to SEQ ID NO:2, a deletion of amino acid residues 2-7 corresponding to SEQ ID NO:2, and a mutation of the fifth cysteine from its N-terminus to a serine corresponding to the cysteine at position 171 of SEQ ID NO:2. The polypeptide sequence of SEQ ID NO:14 was previously described, for example, in U.S. Pat. No. 7,157,559 and WO 07/041,713. The IL-29 polypeptides of the present invention include, for example, SEQ ID NOs:4, 6, 8, 10 and 12, which are encoded by IL-29 polynucleotide molecules as shown in SEQ ID NOs:3, 5, 7, 9 and 11, respectively. The IL-29 polypeptides of the present invention include an amino-terminus truncated IL-29 polypeptide, denoted IL-29 N1 (SEQ ID NO:4—corresponding amino acid residues 1-18 of SEQ ID NO:2 are deleted, and the corresponding cysteines at amino acid positions 112 and 171 of SEQ ID NO:2 are substituted with a serine); a carboxy-terminus truncated IL-29 polypeptide, denoted IL-29 Cl (SEQ ID NO:6—corresponding amino acid residues 1-6 and 168-181 of SEQ ID NO:2 are deleted); a further carboxy-terminus truncated IL-29 polypeptide, denoted IL-29 C2 (SEQ ID NO:8—corresponding amino acid residues 1-6 and 164-181 of SEQ ID NO:2 are deleted); an N1 amino-terminus truncated IL-29 polypeptide and a C1 carboxy-terminus truncated IL-29 polypeptide, denoted as IL-29 N1C1 (SEQ ID NO:10—corresponding amino acid residues 1-18 and 168-181 of SEQ ID NO:2 are deleted, and the cysteine corresponding to amino acid residue 112 of SEQ ID NO:2 is substituted with a serine); and an N1 amino-terminus truncated IL-29 polypeptide and a C2 carboxy-terminus truncated IL-29 polypeptide, denoted as IL-29 N1C2 (SEQ ID NO:12—corresponding amino acid residues 1-18 and 164-181 of SEQ ID NO:2 are deleted, and the cysteine corresponding to amino acid residue 112 is substituted with a serine).

The present invention provides polynucleotide molecules, including DNA and RNA molecules, that encode the IL-29 polypeptides of the present invention. Accordingly, the present invention provides for polynucleotide molecules encoding an IL-29 polypeptide, wherein the encoded IL-29 polypeptide is SEQ ID NO:4, SEQ ID NO:6, SEQ ID NO:8, SEQ ID NO:10 or SEQ ID NO:12. The polypeptide of SEQ ID NO:4, SEQ ID NO:6, SEQ ID NO:8, SEQ ID NO:10 or SEQ ID NO:12, and polynucleotides encoding the same, can include an additional N-terminal methionine (methionine would be at amino acid position one (1) of the polypeptide). The present invention also provides an isolated polypeptide comprising amino acid residues x to 167 of SEQ ID NO:2, wherein x is an integer from 1 to 14. Optionally, the polypeptide comprising amino acid residues x to 167 of SEQ ID NO:2, wherein x is an integer from 1 to 14, may further comprise an N-terminal methionine (methionine would be at amino acid position one (1) of the polypeptide). Those skilled in the art will readily recognize that, in view of the degeneracy of the genetic code, considerable sequence variation is possible among these polynucleotide molecules.

Within one aspect the present invention provides an isolated polypeptide comprising a sequence having at least 90 percent, 91 percent, 92 percent, 93 percent, 94 percent, 95 percent, 96 percent, 97 percent, 98 percent, 99 percent, 99.5 percent or greater than 99.5 percent sequence identity to a sequence of amino acid residues selected from the group consisting of SEQ ID NOs:4, 6, 8, 10 and 12. The polypeptide may optionally comprise at least 15, at least 30, at least 45, or at least 60 sequential amino acids of an amino acid sequence as shown in SEQ ID NOs:4, 6, 8, 10 and 12. In another embodiment, the isolated polypeptide comprises a sequence of amino acid residues of SEQ ID NOs:4, 6, 8, 10 or 12. The polypeptide may have a conservative amino acid change, compared with the amino acid sequence selected from the group consisting of SEQ ID NOs:4, 6, 8, 10 and 12. The polypeptide may further comprise a polyalkyl oxide moiety. The polyalkyl oxide moiety may optionally be polyethylene glycol, such as a 20 kD mPEG propionaldehyde, a 30 kD mPEG propionaldehyde or a 40 kD mPEG propionaldehyde. The polyethylene glycol may be linear or branched. The polyethylene glycol may be covalently attached N-terminally, or covalently attached to the N-terminal methionine, or C-terminally to the polypeptide. Polyalkyl oxide moieties are discussed in the prior art, for example, in U.S. Pat. No. 4,002,531, U.S. Pat. No. 4,179,337, U.S. Pat. No. 4,791,192, U.S. Pat. No. 5,252,714, U.S. Pat. No. 6,774,180 and J. M. Harris, J. Polymer Sci. Polymer Chem. Ed., 23:341 (1984), all of which are herein incorporated by reference in their entirety.

The present invention also provides for a fusion protein comprising a polypeptide that comprises a sequence of amino acid residues selected from the group consisting of SEQ ID NOs:4, 6, 8, 10 and 12; and a polyalkyl oxide moiety. The polyalkyl oxide moiety may optionally be polyethylene glycol, such as a 20 kD mPEG propionaldehyde, a 30 kD mPEG propionaldehyde or a 40 kD mPEG propionaldehyde. The polyethylene glycol may be linear or branched. The polyethylene glycol may be covalently attached N-terminally or C-terminally to the polypeptide. Other polyalkyl oxide moieties that can be covalenty attached to a polypeptide of the present invention are discussed in the prior art, for example, in U.S. Pat. No. 4,002,531, U.S. Pat. No. 4,179,337, U.S. Pat. No. 4,791,192, U.S. Pat. No. 5,252,714, U.S. Pat. No. 6,774,180 and J. M. Harris, J. Polymer Sci. Polymer Chem. Ed., 23:341 (1984), are all of which are herein incorporated by reference for the polyalkyl oxide moieties and the means to covalently attach the polyalkyl oxide moieties to the IL-29 polypeptides of the present invention.

In addition to attaching polyalkyl oxide moieties to the IL-29 polypeptides, human albumin can be genetically coupled to a polypeptide of the present invention to prolong its half-life. Human albumin is the most prevalent naturally occurring blood protein in the human circulatory system, persisting in circulation in the body for over twenty days. Research has shown that therapeutic proteins genetically fused to human albumin have longer half-lives. An IL28 or IL29 albumin fusion protein, like pegylation, may provide patients with long-acting treatment options that offer a more convenient administration schedule, with similar or improved efficacy and safety compared to currently available treatments (U.S. Pat. No. 6,165,470; Syed et al., Blood, 89(9):3243-3253 (1997); Yeh et al., Proc. Natl. Acad. Sci. USA, 89:1904-1908 (1992); and Zeisel et al., Horm. Res., 37:5-13 (1992)).

Like the aforementioned polyalkyl oxide moieties and human albumin, an Fc portion of the human IgG molecule can be fused to a polype1ptide of the present invention. The resultant fusion protein may have an increased circulating half-life due to the Fc moiety (U.S. Pat. No. 5,750,375, U.S. Pat. No. 5,843,725, U.S. Pat. No. 6,291,646; Barouch et al., Journal of Immunology, 61:1875-1882 (1998); Barouch et al., Proc. Natl. Acad. Sci. USA, 97(8):4192-4197 (Apr. 11, 2000); and Kim et al., Transplant Proc., 30(8):4031-4036 (December 1998)).

The present invention also provides for a fusion protein comprising a first polypeptide and a second polypeptide joined by a peptide bond, wherein the first polypeptide comprises a sequence of amino acid residues selected from the group consisting of SEQ ID NOs:4, 6, 8, 10 and 12; and a second polypeptide. The second polypeptide may optionally be a polypeptide selected from the group consisting of affinity tags, toxins, radionucleotides, enzymes and fluorophores.

Table 1 sets forth the one-letter codes to denote degenerate nucleotide positions. “Resolutions” are the nucleotides denoted by a code letter. “Complement” indicates the code for the complementary nucleotide(s). For example, the code Y denotes either C or T, and its complement R denotes A or G, with A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

Degenerate codons encompassing all possible codons for a given amino acid are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence, for example, of SEQ ID NOs:4, 6, 8, 10 and 12. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. A degenerate codon sequence serves as a template for optimizing expression of polynucleotides in various cell types and species commonly used in the art and disclosed herein. Sequences containing preferential codons can be tested and optimized for expression in various species, and tested for functionality as disclosed herein.

Within another aspect, the present invention provides an isolated polynucleotide selected from the group consisting of SEQ ID NOs:3, 5, 7, 9 and 11.

Within another aspect, the present invention provides an isolated polynucleotide capable of hybridizing to a sequence selected from the group consisting of SEQ ID NOs:3, 5, 7, 9 and 11, or a complement thereof, under hybridization conditions of 50% formamide, 5×SSC (1×SSC: 0.15 M sodium chloride and 15 mM sodium citrate), 50 mM sodium phosphate (pH 7.6), 5×Denhardt's solution (100×Denhardt's solution: 2% (w/v) Ficoll 400, 2% (w/v) polyvinylpyrrolidone, and 2% (w/v) bovine serum albumin, 10% dextran sulfate, and 20 mg/ml denatured, sheared salmon sperm DNA at about 42° C. to about 70° C., wherein the isolated polynucleotide encodes a polypeptide having antiviral activity (e.g., inhibits hepatitis C replication), treats multiple sclerosis or any other disease or disorder as disclosed herein. Optionally, the encoded polypeptide has antiviral activity to hepatitis B and/or hepatitis C. Optionally, the isolated polynucleotide may encode at least a portion of a polypeptide sequence selected from the group of SEQ ID NOs:4, 6, 8, 10 and 12. The isolated polynucleotide may encode a polypeptide comprising SEQ ID NOs:4, 6, 8, 10 and 12.

In another aspect, the present invention provides an isolated polynucleotide encoding a polypeptide wherein the encoded polypeptide is selected from the group consisting of SEQ ID NOs:4, 6, 8, 10 and 12.

In another aspect, the present invention provides an isolated polynucleotide encoding a polypeptide wherein the encoded polypeptide has at least 90 percent, 91 percent, 92 percent, 93 percent, 94 percent, 95 percent, 96 percent, 97 percent, 98 percent, 99 percent, 99.5 percent or greater than 99.5 percent sequence identity to a sequence selected from the group consisting of SEQ ID NOs:4, 6, 8, 10 and 12, wherein the encoded polypeptide has antiviral activity (e.g., inhibits hepatitis C replication), treats multiple sclerosis or any other disease or disorder as disclosed herein. Optionally, the encoded polypeptide has antiviral activity is inhibiting hepatitis B and/or hepatitis C replication.

As previously noted, the isolated polynucleotides of the present invention include DNA and RNA. Methods for preparing DNA and RNA are well known in the art. In general, RNA is isolated from a tissue or cell that produces large amounts of IL-28 or IL-29 RNA. Such tissues and cells are identified by Northern blotting (Thomas, Proc. Natl. Acad. Sci. USA 77:5201, 1980), or by screening conditioned medium from various cell types for activity on target cells or tissue. Once the activity or RNA producing cell or tissue is identified, total RNA can be prepared using guanidinium isothiocyanate extraction followed by isolation by centrifugation in a CsCl gradient (Chirgwin et al., Biochemistry 18:52-94, 1979). Poly (A)+ RNA is prepared from total RNA using the method of Aviv and Leder (Proc. Natl. Acad. Sci. USA 69:1408-12, 1972). Complementary DNA (cDNA) is prepared from poly(A)+ RNA using known methods. In the alternative, genomic DNA can be isolated. Polynucleotides encoding IL-29 polypeptides of the present invention are then identified and isolated by, for example, hybridization or PCR.

A full-length clones encoding IL-29 polypeptides of the present invention can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to IL-28 receptor fragments, or other specific binding partners.

Within embodiments of the invention, IL-29 polynucleotides can hybridize under stringent conditions to nucleic acid molecules having the nucleotide sequence of SEQ ID NOs:3, 5, 7, 9 and 11, or to nucleic acid molecules having a nucleotide sequence complementary to SEQ ID NOs:3, 5, 7, 9 and 11. In general, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tm of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases.

It is well within the abilities of one skilled in the art to adapt these conditions for use with a particular polynucleotide hybrid. The Tm for a specific target sequence is the temperature (under defined conditions) at which 50% of the target sequence will hybridize to a perfectly matched probe sequence. Those conditions which influence the Tm include, the size and base pair content of the polynucleotide probe, the ionic strength of the hybridization solution, and the presence of destabilizing agents in the hybridization solution. Numerous equations for calculating Tm are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tm based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tm. For smaller probes, <50 base pairs, hybridization is typically carried out at the Tm or 5-10° C. below the calculated Tm. This allows for the maximum rate of hybridization for DNA-DNA and DNA-RNA hybrids.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding an IL-29 polypeptide of the present invention hybridize with a nucleic acid molecule having the nucleotide sequence of SEQ ID NOs:3, 5, 7, 9 and 11, respectively (or its full-length complement) under stringent washing conditions, in which the wash stringency is equivalent to 0.5×-2×SSC with 0.1% SDS at 55-65° C., including 0.5×SSC with 0.1% SDS at 55° C., or 2×SSC with 0.1% SDS at 65° C. One of skill in the art can readily devise equivalent conditions, for example, by substituting SSPE for SSC in the wash solution.

Typical highly stringent washing conditions include washing in a solution of 0.1×-0.2×SSC with 0.1% sodium dodecyl sulfate (SDS) at 50-65° C. In other words, nucleic acid molecules encoding an IL-29 polypeptide hybridize with a nucleic acid molecule having the nucleotide sequence of SEQ ID NOs:3, 5, 7, 9 and 11 (or its full-length complement) under highly stringent washing conditions, in which the wash stringency is equivalent to 0.1×-0.2×SSC with 0.1% SDS at 50-65° C., including 0.1×SSC with 0.1% SDS at 50° C., or 0.2×SSC with 0.1% SDS at 65° C.

The present invention also provides isolated IL-29 polypeptides that have a substantially similar sequence identity to the polypeptides of the present invention, for example SEQ ID NOs:4, 6, 8, 10 and 12. The term “substantially similar sequence identity” is used herein to denote polypeptides comprising at least 80%, at least 90%, at least 91%, at least 92%, at least 93%, at least 94%, at least 95%, at least 96%, at least 97%, at least 98%, at least 99%, at least 99.5%, or greater than 99.5% sequence identity to the sequences shown in SEQ ID NOs:4, 6, 8, 10 and 12. The present invention also includes IL-29 polypeptides that comprise an amino acid sequence having at least 80%, at least 90%, at least 91%, at least 92%, at least 93%, at least 94%, at least 95%, at least 96%, at least 97%, at least 98%, at least 99%, at least 99.5% or greater than 99.5% sequence identity to a polypeptide selected from the group consisting of SEQ ID NOs:4, 6, 8, 10 and 12. The present invention further includes polynucleotides that encode such polypeptides. The IL-29 polypeptides of the present invention are preferably recombinant polypeptides. In another aspect, the IL-29 polypeptides of the present invention have at least 15, at least 30, at least 45, or at least 60 sequential amino acids. For example, an IL-29 polypeptide of the present invention relates to a polypeptide having at least 15, at least 30, at least 45, or at least 60 sequential amino acids from SEQ ID NOs:4, 6, 8, 10 or 12. Methods for determining percent identity are described below.

The present invention also contemplates variant nucleic acid molecules that can be identified using two criteria: a determination of the similarity between the encoded polypeptide with the amino acid sequence of SEQ ID NOs:4, 6, 8, 10 or 12 respectively, and/or a hybridization assay, as described above. Such variants include nucleic acid molecules: (1) that hybridize with a nucleic acid molecule having the nucleotide sequence of SEQ ID NOs:3, 5, 7, 9 and 11, respectively (or its complement) under stringent washing conditions, in which the wash stringency is equivalent to 0.5×-2×SSC with 0.1% SDS at 55-65° C.; or (2) that encode a polypeptide having at least 80%, at least 90%, at least 91%, at least 92%, at least 93%, at least 94%, at least 95%, at least 96%, at least 97%, at least 98%, at least 99%, at least 99.5% or greater than 99.5% sequence identity to the amino acid sequence of SEQ ID NOs:4, 6, 8, 10 or 12. Alternatively, variants can be characterized as nucleic acid molecules: (1) that hybridize with a nucleic acid molecule having the nucleotide sequence of SEQ ID NOs:3, 5, 7, 9 or 11, respectively (or its complement) under highly stringent washing conditions, in which the wash stringency is equivalent to 0.1×-0.2×SSC with 0.1% SDS at 50-65° C.; and (2) that encode a polypeptide having at least 80%, at least 90%, at least 91%, at least 92%, at least 93%, at least 94%, at least 95%, at least 96%, at least 97%, at least 98%, at least 99%, or greater than 99% sequence identity to the amino acid sequence of SEQ ID NOs:4, 6, 8, 10 or 12, respectively.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 3 (amino acids are indicated by the standard one-letter codes).

$$\frac{Total number of identical matches}{[ \begin{matrix}length of the longer sequence plus the \\ number of gaps introduced into the longer \\ sequence in order to align the two sequences\end{matrix} ]} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant IL-29. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:4) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

IL-29 polypeptides of the present invention with substantially similar sequence identity are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides having at least 80%, at least 90%, at least 91%, at least 92%, at least 93%, at least 94%, at least 95%, at least 96%, at least 97%, at least 98%, at least 99%, at least 99.5% or greater than 99.5% sequence identity to the corresponding region of SEQ ID NOs:4, 6, 8, 10 or 12. Polypeptides comprising affinity tags can further comprise a proteolytic cleavage site between the IL-29 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Determination of amino acid residues that comprise regions or domains that are critical to maintaining structural integrity can be determined. Within these regions one can determine specific residues that will be more or less tolerant of change and maintain the overall tertiary structure of the molecule. Methods for analyzing sequence structure include, but are not limited to alignment of multiple sequences with high amino acid or nucleotide identity, secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in IL-29 polypeptides so as to minimize disruption of higher order structure essential to biological activity. For example, where the IL-29 polypeptide comprises one or more helices, changes in amino acid residues will be made so as not to disrupt the helix geometry and other components of the molecule where changes in conformation abate some critical function, for example, binding of the molecule to its binding partners. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same cysteine pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structurally similarities between proteins and polypeptides (Schaanan et al., Science 257:961-964, 1992).

A Hopp/Woods hydrophilicity profile of the IL-29 polypeptide sequence as shown in SEQ ID NOs:4, 6, 8, 10 or 12 can be generated (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. Those skilled in the art will recognize that hydrophilicity or hydrophobicity will be taken into account when designing modifications in the amino acid sequence of an IL-29 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp.

The identities of essential amino acids can also be inferred from analysis of sequence similarity between IFN-α and IL-29. Using methods such as “FASTA” analysis described previously, regions of high similarity are identified within a family of proteins and used to analyze amino acid sequence for conserved regions. An alternative approach to identifying a variant polynucleotide on the basis of structure is to determine whether a nucleic acid molecule encoding an IL-29 gene can hybridize to a nucleic acid molecule as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl. Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant molecules are tested for biological or biochemical activity as disclosed below to identify amino acid residues that are critical to the activity of the molecule. See also, Hilton et al., J. Biol. Chem. 271:4699 (1996).

The IL-29 polypeptides of the present invention, including full-length polypeptides, biologically active fragments, and fusion polypeptides which can be produced according to conventional techniques using cells into which have been introduced an expression vector encoding the polypeptide. As used herein, “cells into which have been introduced an expression vector” include both cells that have been directly manipulated by the introduction of exogenous DNA molecules and progeny thereof that contain the introduced DNA. Suitable host cells are those cell types that can be transformed or transfected with exogenous DNA and grown in culture, and include bacteria, fungal cells, and cultured higher eukaryotic cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

Within another aspect, the present invention provides an expression vector comprising the following operably linked elements: a transcription promoter; a DNA segment encoding an IL-29 polypeptide of the present invention, e.g., SEQ ID NOs:4, 6, 8, 10 and 12); and a transcription terminator.

The present invention also provides an expression vector comprising an isolated and purified DNA molecule including the following operably linked elements: a transcription promoter; a DNA segment encoding a polypeptide having at least 80%, at least 90%, at least 91%, at least 92%, at least 93%, at least 94%, at least 95%, at least 96%, at least 97%, at least 98%, at least 99%, at least 99.5% or greater than 99.5% sequence identity with a polypeptide selected from the group consisting of SEQ ID NOs:4, 6, 8, 10 and 12; and a transcription terminator. The DNA molecule may further comprise a secretory signal sequence operably linked to the DNA segment. The DNA segment may further comprise an affinity tag operably linked to the DNA segment. The present invention also provides a cultured cell containing the above-described expression vector. The encoded polypeptide may optionally comprise at least 15, at least 30, at least 45, or at least 60 sequential amino acids of an amino acid sequence as shown in SEQ ID NOs:4, 6, 8, 10 and 12. The encoded polypeptide may optionally have antiviral activity, e.g., hepatitis B and hepatitis C, treat multiple sclerosis or any other disease or disorder as disclosed herein.

Within another aspect the present invention provides a cultured cell comprising an expression vector as disclosed herein.

Within another aspect the present invention provides a method of producing a polypeptide comprising: culturing a cell as disclosed above under conditions wherein the DNA segment is expressed; and recovering the polypeptide encoded by the DNA segment.

In general, a DNA sequence encoding an IL-29 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct an IL-29 polypeptide into the secretory pathway of a host cell, a secretory signal sequence (also known as a leader sequence, prepro sequence or pre sequence) is provided in the expression vector. The secretory signal sequence can be SEQ ID NOs:119 or 121 of U.S. Pat. No. 7,157,559, amino acid residues 1-21 of SEQ ID NO:2 or SEQ ID NO:7 of U.S. Pat. No. 7,038,032, or may be derived from another secreted protein (e.g., t-PA; see, U.S. Pat. No. 5,641,655) or synthesized de novo. The secretory signal sequence is operably linked to the IL-29 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

A wide variety of suitable recombinant host cells includes, but is not limited to, gram-negative prokaryotic host organisms. Suitable strains of E. coli include W3110 (genotype F-IN(rrnD-rrnE)1 lambda-), K12-derived strains MM294, TG-1, JM-107, BL21, ZGOLD1 (genotype [F-IN(rrnD-rrnE)1 lambda-ΔompT::tet] is an ΔompT mutant derived from W3110; see U.S. Patent Publication No. 2008/0096252, which is herein incorporated by reference for make and using ZGOLD1), ZGOLD5 (genotype [F-IN(rrnD-rrnE)1 lambda-ΔompT::tet ΔfhuA::Cm] is a ΔfhuA mutant derived from ZGOLD1 (W3110); see U.S. Patent Publication No. 2008/0096252, which is herein incorporated by reference for make and using ZGOLD5) and UT5600. Other suitable strains include: BL21(DE3), BL21(DE3)pLysS, BL21(DE3)pLysE, DH1, DH4I, DH5, DH5I, DH51F′, DH51MCR, DH10B, DH10B/p3, DH11S, C600, HB101, JM101, JM105, JM109, JM110, K38, RR1, Y1088, Y1089, CSH18, ER1451, ER1647, Escherichia coli (E. coli) K12, E. coli K12 RV308, E. coli K12 C600, E. coli HB101, E. coli K12 C600 R.sub.k-M.sub.k-, E. coli K12 RR1 (see, for example, Brown (ed.), Molecular Biology Labfax (Academic Press 1991)). Other gram-negative prokaryotic hosts can include Serratia, Pseudomonas, Caulobacter. Prokaryotic hosts can include gram-positive organisms such as Bacillus, for example, B. subtilis and B. thuringienesis, and B. thuringienesis var. israelensis, as well as Streptomyces, for example, S. lividans, S. ambofaciens, S. fradiae, and S. griseofuscus. Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)). Standard techniques for propagating vectors in prokaryotic hosts are well-known to those of skill in the art (see, for example, Ausubel et al. (eds.), Short Protocols in Molecular Biology, 3rd Edition (John Wiley & Sons 1995); Wu et al., Methods in Gene Biotechnology (CRC Press, Inc. 1997)).

When large scale production of IL-29 using the expression system of the present invention is required, batch fermentation can be used. Generally, batch fermentation comprises that a first stage seed flask is prepared by growing E. coli strains expressing IL-29 in a suitable medium in shake flask culture to allow for growth to an optical density (OD) of between 5 and 20 at 600 nm. A suitable medium would contain nitrogen from a source(s) such as ammonium sulfate, ammonium phosphate, ammonium chloride, yeast extract, hydrolyzed animal proteins, hydrolyzed plant proteins or hydrolyzed caseins. Phosphate will be supplied from potassium phosphate, ammonium phosphate, phosphoric acid or sodium phosphate. Other components would be magnesium chloride or magnesium sulfate, ferrous sulfate or ferrous chloride, and other trace elements. Growth medium can be supplemented with carbohydrates, such as fructose, glucose, galactose, lactose, and glycerol, to improve growth. Alternatively, a fed batch culture is used to generate a high yield of IL-29 protein. The IL-29 producing E. coli strains are grown under conditions similar to those described for the first stage vessel used to inoculate a batch fermentation.

Following fermentation the cells are harvested by centrifugation, re-suspended in homogenization buffer and homogenized, for example, in an APV-Gaulin homogenizer (Invensys APV, Tonawanda, N.Y.) or other type of cell disruption equipment, such as bead mills or sonicators. Alternatively, the cells are taken directly from the fermentor and homogenized in an APV-Gaulin homogenizer. The washed inclusion body prep can be solubilized using guanidine hydrochloride (5-8 M) or urea (7-8 M) containing a reducing agent such as beta mercaptoethanol (10-100 mM) or dithiothreitol (5-50 mM). The solutions can be prepared in Tris, phopshate, HEPES or other appropriate buffers. Inclusion bodies can also be solubilized with urea (2-4 M) containing sodium lauryl sulfate (0.1-2%). In the process for recovering purified IL-29 from transformed E. coli host strains in which the IL-29 is accumulates as refractile inclusion bodies, the cells are disrupted and the inclusion bodies are recovered by centrifugation. The inclusion bodies are then solubilized and denatured in 6 M guanidine hydrochloride containing a reducing agent. The reduced IL-29 is then oxidized in a controlled renaturation step. Refolded IL-29 can be passed through a filter for clarification and removal of insoluble protein. The solution is then passed through a filter for clarification and removal of insoluble protein. After the IL-29 protein is refolded and concentrated, the refolded IL-29 protein is captured in dilute buffer on a cation exchange column and purified using hydrophobic interaction chromatography.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993), and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant baculovirus utilizes a transposon-based system described by Luckow (Luckow, V. A, et al., J Virol 67:4566-79, 1993). This system is sold in the Bac-to-Bac kit (Life Technologies, Rockville, Md.). This system utilizes a transfer vector, pFastBac1 (Life Technologies) containing a Tn7 transposon to move the DNA encoding the IL-29 polypeptide into a baculovirus genome maintained in E. coli as a large plasmid called a “bacmid.” The pFastBac1 transfer vector utilizes the AcNPV polyhedrin promoter to drive the expression of the gene of interest, in this case IL-29. However, pFastBac1 can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S, and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native IL-29 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native IL-29 secretory signal sequence. In addition, transfer vectors can include an in-frame fusion with DNA encoding an epitope tag at the C- or N-terminus of the expressed mutant IL-29 polypeptide, for example, a Glu-Glu epitope tag (Grussenmeyer, T. et al., Proc. Natl. Acad. Sci. 82:7952-4, 1985). Using techniques known in the art, a transfer vector containing IL-29 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses IL-29 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology Principles and Applications of Recombinant DNA, ASM Press, Washington, D.C., 1994. Another suitable cell line is the High FiveO* cell line (Invitrogen) derived from Trichoplusia ni (U.S. Pat. No. 5,300,435).

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. The use of Pichia methanolica as host for the production of recombinant proteins is disclosed in U.S. Pat. Nos. 5,955,349, 5,888,768 and 6,001,597, U.S. Pat. No. 5,965,389, U.S. Pat. No. 5,736,383, and U.S. Pat. No. 5,854,039.

It is preferred to purify the polypeptides and proteins of the present invention to 80% purity, more preferably to 90% purity, even more preferably 95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide or protein is substantially free of other polypeptides or proteins, particularly those of animal origin.

Expressed recombinant IL-29 proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification Principles and Practice, Springer-Verlag, New York, 1994. Proteins comprising a polyhistidine affinity tag (typically about 6 histidine residues) are purified by affinity chromatography on a nickel chelate resin. See, for example, Houchuli et al., Bio/Technol. 6: 1321-1325, 1988. Proteins comprising a glu-glu tag can be purified by immunoaffinity chromatography according to conventional procedures. See, for example, Grussenmeyer et al., supra. Maltose binding protein fusions are purified on an amylose column according to methods known in the art.

IL-29 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

As used herein, the term “antibodies” includes polyclonal antibodies, monoclonal antibodies, antigen-binding fragments thereof such as F(ab′)₂ and Fab fragments, single chain antibodies, and the like, including genetically engineered antibodies. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. One skilled in the art can generate humanized antibodies with specific and different constant domains (i.e., different Ig subclasses) to facilitate or inhibit various immune functions associated with particular antibody constant domains. Antibodies are defined to be specifically binding if they bind to the IL-29 polypeptide or protein with an affinity of at least 10-fold greater than the binding affinity to control (non mutant IL-29) polypeptide or protein. The affinity of a monoclonal antibody can be readily determined by one of ordinary skill in the art (see, for example, Scatchard, Ann. NY Acad. Sci. 51: 660-672, 1949).

Methods for preparing polyclonal and monoclonal antibodies are well known in the art (see for example, Hurrell, J. G. R., Ed., Monoclonal Hybridoma Antibodies: Techniques and Applications, CRC Press, Inc., Boca Raton, Fla., 1982, which is incorporated herein by reference). The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

A variety of assays known to those skilled in the art can be utilized to detect antibodies which specifically bind to IL-29 polypeptides. Exemplary assays are described in detail in Using Antibodies: A Laboratory Manual, Harlow and Lane (Eds.), Cold Spring Harbor Laboratory Press, 1999. Representative examples of such assays include: concurrent immunoelectrophoresis, radio-immunoassays, radio-immunoprecipitations, enzyme-linked immunosorbent assays (ELISA), dot blot assays, Western blot assays, inhibition or competition assays, and sandwich assays.

For certain applications, including in vitro and in vivo diagnostic uses, it is advantageous to employ labeled antibodies. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies of the present invention may also be directly or indirectly conjugated to drugs, toxins, radionuclides and the like, and these conjugates used for in vivo diagnostic or therapeutic applications (e.g., inhibition of cell proliferation). See, in general, Ramakrishnan et al., Cancer Res. 56:1324-1330, 1996.

Using methods known in the art, IL-29 polypeptides can be prepared as monomers or multimers; glycosylated or non-glycosylated; pegylated or non-pegylated; fusion proteins; and may or may not include an initial methionine amino acid residue. IL-29 conjugates used for therapy may comprise pharmaceutically acceptable water-soluble polymer moieties. Conjugation of interferons with water-soluble polymers has been shown to enhance the circulating half-life of the interferon, and to reduce the immunogenicity of the polypeptide (see, for example, Nieforth et al., Clin. Pharmacol. Ther. 59:636 (1996), and Monkarsh et al., Anal. Biochem. 247:434 (1997)).

Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, monomethoxy-PEG propionaldehyde, PEG propionaldehyde, bis-succinimidyl carbonate PEG, propylene glycol homopolymers, a polypropylene oxide/ethylene oxide co-polymer, polyoxyethylated polyols (e.g., glycerol), monomethoxy-PEG butyraldehyde, PEG butyraldehyde, monomethoxy-PEG acetaldehyde, PEG acetaldehyde, methoxyl PEG-succinimidyl propionate, methoxyl PEG-succinimidyl butanoate, polyvinyl alcohol, dextran, cellulose, or other carbohydrate-based polymers. Suitable PEG may have a molecular weight from about 600 to about 60,000, including, for example, 5,000 daltons, 12,000 daltons, 20,000 daltons, 30,000 daltons, and 40,000 daltons, which can be linear or branched. An IL-29 conjugate or fusion protein can also comprise a mixture of such water-soluble polymers.

One example of an IL-29 conjugate or fusion protein comprises an IL-29 moiety and a polyalkyl oxide moiety attached, for example, to the N-terminus of the IL-29 polypeptide. Polyethylene glycol (“PEG”) is one suitable polyalkyl oxide moiety. As an illustration, IL-29 can be modified with PEG, a process known as “PEGylation.” PEGylation of IL-29 can be carried out by any of the PEGylation reactions known in the art (see, for example, EP 0 154 316, Delgado et al., Critical Reviews in Therapeutic Drug Carrier Systems 9:249 (1992), Duncan and Spreafico, Clin. Pharmacokinet 27:290 (1994), and Francis et al., Int J Hematol 68:1 (1998)). For example, PEGylation can be performed by an acylation reaction or by an alkylation reaction with a reactive polyethylene glycol molecule. In an alternative approach, IL-29 conjugates are formed by condensing activated PEG, in which a terminal hydroxy or amino group of PEG has been replaced by an activated linker (see, for example, Karasiewicz et al., U.S. Pat. No. 5,382,657).

PEGylation by acylation typically requires reacting an active ester derivative of PEG with an IL-29 polypeptide. An example of an activated PEG ester is PEG esterified to N-hydroxysuccinimide. As used herein, the term “acylation” includes the following types of linkages between IL-29 and a water-soluble polymer: amide, carbamate, urethane, and the like. Methods for preparing PEGylated IL-29 by acylation will typically comprise the steps of (a) reacting an IL-29 polypeptide with PEG (such as a reactive ester of an aldehyde derivative of PEG) under conditions whereby one or more PEG groups attach to IL-29, and (b) obtaining the reaction product(s). Generally, the optimal reaction conditions for acylation reactions will be determined based upon known parameters and desired results. For example, the larger the ratio of PEG: IL-29, the greater the percentage of polyPEGylated IL-29 product.

PEGylation by alkylation generally involves reacting a terminal aldehyde, e.g., propionaldehyde, butyraldehyde, acetaldehyde, and the like, derivative of PEG with IL-29 in the presence of a reducing agent. PEG groups are preferably attached to the polypeptide via a —CH2-NH2 group.

Derivatization via reductive alkylation to produce a monoPEGylated product takes advantage of the differential reactivity of different types of primary amino groups available for derivatization. Typically, the reaction is performed at a pH that allows one to take advantage of the pKa differences between the ε-amino groups of the lysine residues and the α-amino group of the N-terminal residue of the protein. By such selective derivatization, attachment of a water-soluble polymer that contains a reactive group such as an aldehyde, to a protein is controlled. The conjugation with the polymer occurs predominantly at the N-terminus of the protein without significant modification of other reactive groups such as the lysine side chain amino groups.

Reductive alkylation to produce a substantially homogenous population of monopolymer IL-29 conjugate molecule can comprise the steps of: (a) reacting a IL-29 polypeptide with a reactive PEG under reductive alkylation conditions at a pH suitable to permit selective modification of the α-amino group at the amino terminus of the IL-29, and (b) obtaining the reaction product(s). The reducing agent used for reductive alkylation should be stable in aqueous solution and preferably be able to reduce only the Schiff base formed in the initial process of reductive alkylation. Preferred reducing agents include sodium borohydride, sodium cyanoborohydride, dimethylamine borane, trimethylamine borane, and pyridine borane.

For a population of monopolymer IL-29 conjugates, the reductive alkylation reaction conditions are those that permit the selective attachment of the water-soluble polymer moiety to the N-terminus of IL-29. Such reaction conditions generally provide for pKa differences between the lysine amino groups and the α-amino group at the N-terminus. The pH also affects the ratio of polymer to protein to be used. In general, if the pH is lower, a larger excess of polymer to protein will be desired because the less reactive the N-terminal α-group, the more polymer is needed to achieve optimal conditions. If the pH is higher, the polymer: Cysteine IL-29 need not be as large because more reactive groups are available. Typically, the pH will fall within the range of 3-9, or 3-6. Another factor to consider is the molecular weight of the water-soluble polymer. Generally, the higher the molecular weight of the polymer, the fewer number of polymer molecules which may be attached to the protein. For PEGylation reactions, the typical molecular weight is about 2 kDa to about 100 kDa, about 5 kDa to about 50 kDa, about 12 kDa to about 40 kDa, or about 20 kDa to about 30 kDa. The molar ratio of water-soluble polymer to IL-29 will generally be in the range of 1:1 to 100:1. Typically, the molar ratio of water-soluble polymer to mutant IL-29 will be 1:1 to 20:1 for polyPEGylation, and 1:1 to 5:1 for monoPEGylation.

General methods for producing conjugates comprising interferon and water-soluble polymer moieties are known in the art. See, for example, Karasiewicz et al., U.S. Pat. No. 5,382,657, Greenwald et al., U.S. Pat. No. 5,738,846, Nieforth et al., Clin. Pharmacol. Ther. 59:636 (1996), Monkarsh et al., Anal. Biochem. 247:434 (1997). PEGylated species can be separated from unconjugated mutant IL-29 polypeptides using standard purification methods, such as dialysis, ultrafiltration, ion exchange chromatography, affinity chromatography, size exclusion chromatography, and the like.

The IL-29 molecules of the present invention are capable of specifically binding the IL-28 receptor and/or acting as an antiviral agent. The binding of IL-29 polypeptides to the IL-28 receptor can be assayed using established approaches. IL-29 can be iodinated using an iodobead (Pierce, Rockford, Ill.) according to manufacturer's directions, and the ¹²⁵I-IL-28 or ¹²⁵I-IL-29 can then be used as described below.

In a first approach fifty nanograms of ¹²⁵I-IL-29 can be combined with 1000 ng of IL-28 receptor human IgG fusion protein, in the presence or absence of possible binding competitors including unlabeled IL-29. The same binding reactions would also be performed substituting other cytokine receptor human IgG fusions as controls for specificity. Following incubation at 4° C., protein-G (Zymed, SanFransisco, Calif.) is added to the reaction, to capture the receptor-IgG fusions and any proteins bound to them, and the reactions are incubated another hour at 4° C. The protein-G sepharose is then collected, washed three times with PBS and ¹²⁵I-IL-29 bound is measure by gamma counter (Packard Instruments, Downers Grove, Ill.).

In a second approach, the ability of molecules to inhibit the binding of ¹²⁵I-IL-29 to plate bound receptors can be assayed. A fragment of the IL-28 receptor, representing the extracellular, ligand binding domain, can be adsorbed to the wells of a 96 well plate by incubating 100 μl of 1 g/mL solution of receptor in the plate overnight. In a second form, a receptor-human IgG fusion can be bound to the wells of a 96 well plate that has been coated with an antibody directed against the human IgG portion of the fusion protein. Following coating of the plate with receptor the plate is washed, blocked with SUPERBLOCK (Pierce, Rockford, Ill.) and washed again. Solutions containing a fixed concentration of ¹²⁵I-IL-29 with or without increasing concentrations of potential binding competitors including IL-29, and 100 μl of the solution added to appropriate wells of the plate. Following a one-hour incubation at 4° C. the plate is washed and the amount ¹²⁵I-IL-29 bound determined by counting (Topcount, Packard Instruments, Downers grove, IL). The specificity of binding of ¹²⁵I-IL-29 can be defined by receptor molecules used in these binding assays as well as by the molecules used as inhibitors.

In addition to pegylation, human albumin can be genetically coupled to a polypeptide of the present invention to prolong its half-life. Human albumin is the most prevalent naturally occurring blood protein in the human circulatory system, persisting in circulation in the body for over twenty days. Research has shown that therapeutic proteins genetically fused to human albumin have longer half-lives. An IL29 albumin fusion protein, like pegylation, may provide patients with long-acting treatment options that offer a more convenient administration schedule, with similar or improved efficacy and safety compared to currently available treatments (U.S. Pat. No. 6,165,470; Syed et al., Blood, 89(9):3243-3253 (1997); Yeh et al., Proc. Natl. Acad. Sci. USA, 89:1904-1908 (1992); and Zeisel et al., Horm. Res., 37:5-13 (1992)).

Like the aforementioned peglyation and human albumin, an Fc portion of the human IgG molecule can be fused to a polypeptide of the present invention. The resultant fusion protein may have an increased circulating half-life due to the Fc moiety (U.S. Pat. No. 5,750,375, U.S. Pat. No. 5,843,725, U.S. Pat. No. 6,291,646; Barouch et al., Journal of Immunology, 61:1875-1882 (1998); Barouch et al., Proc. Natl. Acad. Sci. USA, 97(8):4192-4197 (Apr. 11, 2000); and Kim et al., Transplant Proc., 30(8):4031-4036 (December 1998)).

There are several in vivo models for testing HBV and HCV that are known to those skilled in art. With respect to HCV, for example, the HCV Replicon model is a cell-based system to study the effectiveness of a drug to inhibit HCV replication (Blight et al., Science, 290(5498):1972-1974 (Dec. 8, 2000); and Lohmann et al., Science, 285(5424):110-113 (Jul. 2, 1999)). A well-known and accepted in vitro HBV model to one of skill in the art can be used to determine the anti-HBV activity of a test molecule is disclosed in Korba et al., Antiviral Res., 19(1):55-70 (1992) and Korba et al., Antiviral Res., 15(3):217-228 (1991).

For example, the effects of IL-29 on mammals infected with HBV can accessed using a woodchuck model. Briefly, woodchucks chronically infected with woodchuck hepatitis virus (WHV) develop hepatitis and hepatocellular carcinoma that is similar to disease in humans chronically infected with HBV. The model has been used for the preclinical assessment of antiviral activity. A chronically infected WHV strain has been established and neonates are inoculated with serum to provide animals for studying the effects of certain compounds using this model. (For a review, see, Tannant et al., ILAR J. 42 (2):89-102, 2001). Chimpanzees may also be used to evaluate the effect of IL-29 on HBV infected mammals. Using chimpanzees, characterization of HBV was made and these studies demonstrated that the chimpanzee disease was remarkably similar to the disease in humans (Barker et al., J. Infect. Dis. 132:451-458, 1975 and Tabor et al., J. Infect. Dis. 147:531-534, 1983.) The chimpanzee model has been used in evaluating vaccines (Prince et al., In: Vaccines 97, Cold Spring Harbor Laboratory Press, 1997.) Therapies for HIV are routinely tested using non-human primates infected with simian immunodeficiency viruses (for a review, see, Hirsch et al., Adv. Pharmcol. 49:437-477, 2000 and Nathanson et al., AIDS13 (suppl. A):5113-5120, 1999.) For a review of use of non-human primates in HIV, hepatitis, malaria, respiratory syncytial virus, and other diseases, see, Sibal et al., ILAR J. 42 (2):74-84, 2001. A recently developed transgenic mouse model (Guidotti et al., Journal of Virology 69:6158-6169, 1995) supports the replication of high levels of infectious HBV and has been used as a chemotherapeutic model for HBV infection. Transgenic mice are treated with antiviral drugs and the levels of HBV DNA and RNA are measured in the transgenic mouse liver and serum following treatment. HBV protein levels can also be measured in the transgenic mouse serum following treatment. This model has been used to evaluate the effectiveness of lamivudine and IFN-alpha in reducing HBV viral titers (Morrey et al., Antiviral Therapy 3:59-68, 1998).

### Uses of the Novel IL-29 Mutants

A. Antiviral Treatment

The present invention also provides for treating a patient having a viral infection comprising administering to the patient a pharmaceutically effective amount of a fusion protein of the present invention, wherein after administration of the fusion protein the viral load has reduced or viral replication is inhibited. Optionally, the viral infection is a result of a virus selected from the group consisting of DNA Viruses (e.g., Herpes Viruses such as Herpes Simplex viruses, Epstein-Barr virus, Cytomegalovirus; Pox viruses such as Variola (small pox) virus; Hepadnaviruses (e.g., Hepatitis B virus); Papilloma viruses; Adenoviruses); RNA Viruses (e.g., HIV I, II; HTLV I, II; Poliovirus; Hepatitis A; Orthomyxoviruses (e.g., Influenza viruses); Paramyxoviruses (e.g., Measles virus); Rabies virus; Hepatitis C); Rhinovirus, Respiratory Syncytial Virus, Vaccinia Virus, Dengue Virus, Cytomegalovirus, West Nile Virus, Yellow Fever, Rift Valley Virus, Lassa Fever Virus, Ebola Virus, Lymphocytic Choriomeningitis Virus, Human Immunodeficienty virus (HIV), viral meningitis, severe acute respiratory syndrome (SARS) coronavirus and HIV-related disease. Optionally, the patient has an Hepatitis B or Hepatitis C infection.

There are several in vivo models for testing HBV and HCV that are known to those skilled in art. With respect to HCV, for example, the HCV Replicon model is a cell-based system to study the effectiveness of a drug to inhibit HCV replication (Blight et al., Science, 290(5498):1972-1974 (Dec. 8, 2000); and Lohmann et al., Science, 285(5424):110-113 (Jul. 2, 1999)), both of which are herein incorporated by reference as relating to the HCV Replicon model. A well-known and accepted in vitro HBV model to one of skill in the art can be used to determine the anti-HBV activity of a test molecule is disclosed in Korba et al., Antiviral Res., 19(1):55-70 (1992) and Korba et al., Antiviral Res., 15(3):217-228 (1991), both of which are herein incorporated by reference as relating to the in vitro HBV model.

B. Cancer Treatment

An IL-29 polypeptide of the present invention can be used to treat any of the following disorders: carcinoma, a sarcoma, a glioma, a lymphoma, a leukemia, or a skin cancer. The carcinoma can be a skin, an esophageal, a gastric, a colonic, a rectal, a pancreatic, a lung, a breast, an ovarian, a urinary bladder, an endometrial, a cervical, a testicular, a renal, an adrenal or a liver carcinoma. B-cell related disease may be an indolent form of B-cell lymphoma, an aggressive form of B-cell lymphoma, non-Hodgkin's lymphoma, a chronic lymphocytic leukemia, an acute lymphocytic leukemia, a Waldenstrom's macroglobulinemia, or a multiple myeloma. In addition, the B-cell related disease can be a human or a veterinary type of disease. Neovascular disorders amenable to treatment in accordance with the present invention include, for example, cancers characterized by solid tumor growth (e.g., pancreatic cancer, renal cell carcinoma (RCC), colorectal cancer, non-small cell lung cancer (NSCLC), and gastrointestinal stromal tumor (GIST)) as well as various neovascular ocular disorders (e.g., age-related macular degeneration, diabetic retinopathy, iris neovascularization, and neovascular glaucoma). A T-cell related disease may be a human or veterinary T-cell leukemia, skin psoriasis, psoriatic arthritis or mycosis fungoides. A metabolic disease can be an amyloidosis. A neurodegenerative disease can be an Alzheimer's disease.

1. Types of Cancer

TABLE 5
Exemplary Cancers Involving Solid Tumor Formation

| | |
|---|---|
| 1. | Head and Neck cancer |

| | |
|---|---|
| a. | Brain |
| b. | Oral cavity |
| c. | Orophyarynx |
| d. | Nasopharynx |
| e. | Hypopharynx |
| f. | Nasal cavities and paranasal sinuses |
| g. | Larynx |
| h. | Lip |

| | |
|---|---|
| 2. | Lung cancers |

| | |
|---|---|
| a. | Non-small cell carcinoma |
| b. | Small cell carcinoma |

| | |
|---|---|
| 3. | Gastrointestinal Tract cancers |

| | |
|---|---|
| a. | Colorectal cancer |
| b. | Gastric cancer |
| c. | Esophageal cancer |
| d. | Anal cancer |
| e. | Extrahepatic Bile Duct cancer |
| f. | Cancer of the Ampulla of Vater |
| g. | Gastrointestinal Stromal Tumor (GIST) |

| | |
|---|---|
| 4. | Liver cancer |

| | |
|---|---|
| a. | Liver Cell Adenoma |
| b. | Hepatocellular Carcinoma |

| | |
|---|---|
| 5. | Breast cancer |
| 6. | Gynecologic cancer |

| | |
|---|---|
| a. | Cervical cancer |
| b. | Ovarian cancer |
| c. | Vaginal cancer |
| d. | Vulvar cancer |
| e. | Gestational Trophoblastic Neoplasia |
| f. | Uterine cancer |

| | |
|---|---|
| 7. | Urinary Tract cancer |

| | |
|---|---|
| a. | Renal cancer carcinoma |
| b. | Prostate cancer |
| c. | Urinary Bladder cancer |
| d. | Penile cancer |
| e. | Urethral cancer |

| | |
|---|---|
| 8. | Urinary Bladder cancer |
| 9. | Neurological Tumors |

| | |
|---|---|
| a. | Astrocytoma and glioblastoma |
| b. | Primary CNS lymphoma |
| c. | Medulloblastoma |
| d. | Germ Cell tumors |
| e. | Retinoblastoma |

| | |
|---|---|
| 10. | Endocrine Neoplasms |

| | |
|---|---|
| a. | Thyroid cancer |
| b. | Pancreatic cancer |

| | |
|---|---|
| 1) | Islet Cell tumors |

| | |
|---|---|
| a) | Insulinomas |
| b) | Glucagonomas |
| c. | Pheochromocytoma |
| d. | Adrenal carcinoma |
| e. | Carcinoid tumors |
| f. | Parathyroid cancinoma |
| g. | Pineal gland neoplasms |

| | |
|---|---|
| 11. | Skin cancers |

| | |
|---|---|
| a. | Malignant melanoma |
| b. | Squamous Cell carcinoma |
| c. | Basal Cell carcinoma |
| d. | Kaposi's Sarcoma |

| | |
|---|---|
| 12. | Bone cancers |

| | |
|---|---|
| a. | Osteoblastoma |
| b. | Osteochondroma |
| c. | Osteosarcoma |

| | |
|---|---|
| 13. | Connective Tissue neoplasms |

| | |
|---|---|
| a. | Chondroblastoma |
| b. | Chondroma |

| | |
|---|---|
| 14. | Hematopoietic malignancies |

| | |
|---|---|
| a. | Non-Hodgkin Lymphoma |

| | |
|---|---|
| 1) | B-cell lymphoma |
| 2) | T-cell lymphoma |
| 3) | Undifferentiated lymphoma |

| | |
|---|---|
| b. | Leukemias |

| | |
|---|---|
| 1) | Chronic Myelogenous Leukemia |
| 2) | Hairy Cell Leukemia |
| 3) | Chronic Lymphocytic Leukemia |
| 4) | Chronic Myelomonocytic Leukemia |
| 5) | Acute Myelocytic Leukemia |
| 6) | Acute Lymphoblastic Leukemia |

| | |
|---|---|
| c. | Myeloproliferative Disorders |

| | |
|---|---|
| 1) | Multiple Myeloma |
| 2) | Essential Thrombocythemia |
| 3) | Myelofibrosis with Myeloid Metaplasia |
| 4) | Hypereosinophilic Syndrome |
| 5) | Chronic Eosinophilic Leukemia |
| 6) | Polycythemia Vera |

| | |
|---|---|
| d. | Hodgkin Lymphoma |

| | |
|---|---|
| 15. | Childhood Cancers |

| | |
|---|---|
| a. | Leukemia and Lymphomas |
| b. | Brain cancers |
| c. | Neuroblastoma |
| d. | Wilm's Tumor (nephroblastoma) |
| e. | Phabdomyosarcoma |
| f. | Retinoblastoma |

| | |
|---|---|
| 16. | Immunotherapeutically sensitive cancers |

| | |
|---|---|
| a. | melanoma |
| b. | kidney cancer |
| c. | leukemias, lymphomas and myelomas |
| d. | breast cancer |
| e. | prostate cancer |
| f. | colorectal cancer |
| g. | cervical cancer |
| h. | ovarian cancer |
| i. | lung cancer |

Some of the cancers listed above, including some of the relevant animal models for evaluating the effects of an IL-29 polypeptide of the present invention on such cancers, are discussed in further detail below.

An IL-29 polypeptide of the present invention can be used to treat chronic myeloid leukemia (CML). CML is a rare type of cancer affecting mostly adults. It is a cancer of granulocytes (one of the main types of white blood cells). In CML many granulocytes are produced and they are released into the blood when they are immature and unable to work properly. The production of other types of blood cells is also disrupted. Normally, white blood cells repair and reproduce themselves in an orderly and controlled manner, but in chronic myeloid leukemia the process gets out of control and the cells continue to divide and mature abnormally. The disease usually develops very slowly, which is why it is called ‘chronic’ myeloid leukemia. Because CML develops (progresses) slowly, it is difficult to detect in its early stages. The symptoms of CML are often vague and non-specific and are caused by the increased number of abnormal white blood cells in the bone marrow and the reduced number of normal blood cells: a feeling of fullness or a tender lump on the left side of the abdomen because of enlargement of the spleen. The effects of an IL-29 polypeptide of the present invention for the treatment of chronic myeloid leukemia can be evaluated in a murine chronic myeloid leukemia model similar to that described in Ren, R., Oncogene. 2002 Dec. 9; 21(56):8629-42; Wertheim et al., Oncogene. 2002 Dec. 9; 21(56):8612-28; and Wolff et al., Blood. 2001 Nov. 1; 98(9):2808-16.

An IL-29 polypeptide of the present invention can be used to treat multiple myeloma. Multiple myeloma is a type of cancer that affects the plasma cells by causing their unregulated production. Myeloma cells tend to collect in the bone marrow and in the hard, outer part of bones. Myeloma cells can form a single mass, or tumor called a plasmacytoma or form many tumors, thus the disease is called multiple myeloma. Those suffering from multiple myeloma have an abnormally large number of identical plasma cells, and also have too much of one type of antibody. These myeloma cells and antibodies can cause a number of serious medical problems: (1) myeloma cells damage and weaken bones, causing pain and sometimes fractures; (2) hypocalcaemia, which often results in loss of appetite, nausea, thirst, fatigue, muscle weakness, restlessness, and confusion; (3) myeloma cells prevent the bone marrow from forming normal plasma cells and other white blood cells that are important to the immune system; (4) myeloma cells prevent the growth of new red blood cells, causing anemia; and (5) kidney problems. Symptoms of multiple myeloma depend on how advanced is the disease. In the earliest stage of the disease a patient may be asymptomatic. Symptoms include bone pain, broken bones, weakness, fatigue, weight loss, repeated infections, nausea, vomiting, constipation, problems with urination, and weakness or numbness in the legs. The effects of an IL-29 polypeptide of the present invention designed to treat multiple myeloma can be evaluated in a multiple myeloma murine model similar to that described in Oyajobi et al., Blood. 2003 Jul. 1; 102(1):311-9; Croucher et al., J Bone Miner Res. 2003 March; 18(3):482-92; Asosingh et al., Hematol J. 2000; 1(5):351-6; and Miyakawa et al., Biochem Biophys Res Commun. 2004 Jan. 9; 313(2):258-62.

An IL-29 polypeptide of the present invention can be used to treat lymphomas. Lymphomas are a type of cancer of the lymphatic system. There are two main types of lymphoma. One is called Hodgkin's disease (named after Dr Hodgkin, who first described it). The other is called non-Hodgkin's lymphoma. There are about 20 different types of non-Hodgkin's lymphoma. In most cases of Hodgkin's disease, a particular cell known as the Reed-Sternberg cell is found in the biopsies. This cell is not usually found in other lymphomas, so they are called non-Hodgkin's lymphoma. Symptoms of a non-Hodgkin's lymphoma is a painless swelling of a lymph node in the neck, armpit or groin; night sweats or unexplained high temperatures (fever); loss of appetite, unexplained weight loss and excessive tiredness. The effects of an IL-29 polypeptide of the present invention designed to treat a lymphoma, particularly a non-Hodgkin's lymphoma, can be evaluated in a murine non-Hodgkin's lymphoma model similar to that described in Ansell et al., Leukemia. 2004 March; 18(3):616-23; De Jonge et al., J. Immunol. 1998 Aug. 1; 161(3):1454-61; and Slavin et al., Nature. 1978 Apr. 13; 272(5654):624-6.

An IL-29 polypeptide of the present invention can be used to treat Non-Hodgkin's lymphoma. The classification of Non-Hodgkin's lymphomas most commonly used is the REAL classification system (Ottensmeier, Chemico-Biological Interactions 135-136:653-664, 2001.) Specific immunological markers have been identified for classifications of lymphomas. For example, follicular lymphoma markers include CD20+, CD3−, CD10+, CD5−; small lymphocytic lymphoma markers include CD20+, CD3−, CD10−, CD5+, CD23+; marginal zone B cell lymphoma markers include CD20+, CD3−, CD10−, CD23−; diffuse large B cell lymphoma markers include CD20+, CD3−; mantle cell lymphoma markers include CD20+, CD3−, CD10−, CD5+, CD23+; peripheral T-cell lymphoma markers include CD20−, CD3+; primary mediastinal large B cell lymphoma markers include CD20+, CD3−, lymphoblastic lymphoma markers include CD20−, CD3+, Tdt+, and Burkitt's lymphoma markers include CD20+, CD3−, CD10+, CD5− (Decision Resourses, Non-Hodgkins Lymphoma, Waltham, Mass., February 2002).

An IL-29 polypeptide of the present invention can be used to treat melanomas. Superficial spreading melanoma is the most common type of melanoma. About 7 out of 10 (70%) are this type. The most common place in women is on the legs, while in men it is more common on the trunk, particularly the back. They tend to start by spreading out across the surface of the skin: this is known as the radial growth phase. The melanoma will then start to grow down deeper into the layers of the skin, and eventually into the bloodstream or lymph system to other parts of the body. Nodular melanoma occurs most often on the chest or back. It tends to grow deeper into the skin quite quickly if it is not removed. This type of melanoma is often raised above the rest of the skin surface and feels like a bump. It may be very dark brown-black or black. Lentigo maligna melanoma is most commonly found on the face. It grows slowly and may take several years to develop. Acral melanoma is usually found on the palms of the hands, soles of the feet or around the toenails. Other very rare types of melanoma of the skin include amelanotic melanoma (in which the melanoma loses its pigment and appears as a white area) and desmoplastic melanoma (which contains fibrous scar tissue). Malignant melanoma can start in parts of the body other than the skin but this is very rare. The parts of the body that may be affected are the eye, the mouth, under the fingernails (known as subungual melanoma) the vulval or vaginal tissues, or internally. The effects of an IL-29 polypeptide of the present invention designed to treat melanoma can be evaluated in a murine melanoma model similar to that described in Hermans et al., Cancer Res. 2003 Dec. 1; 63(23):8408-13; Ramont et al., Exp Cell Res. 2003 Nov. 15; 291(1):1-10; Safwat et al., J Exp Ther Oncol. 2003 July-August; 3(4):161-8; and Fidler, I. J., Nat New Biol. 1973 Apr. 4; 242(118):148-9.

An IL-29 polypeptide of the present invention can be used to treat renal cell carcinoma. Renal cell carcinoma, a form of kidney cancer that involves cancerous changes in the cells of the renal tubule. The first symptom is usually blood in the urine. The cancer metastasizes or spreads easily; most often spreading to the lungs and other organs. The effects of an IL-29 polypeptide of the present invention designed to treat melanoma can be evaluated in a murine renal cell carcinoma model similar to that described in Sayers et al., Cancer Res. 1990 Sep. 1; 50(17):5414-20; Salup et al., Immunol. 1987 Jan. 15; 138(2):641-7; and Luan et al., Transplantation. 2002 May 27; 73(10):1565-72.

An IL-29 polypeptide of the present invention can be used to treat cervical cancer. Cervical cancer, also called cervical carcinoma, develops from abnormal cells on the surface of the cervix. Cervical cancer is usually preceded by dysplasia, precancerous changes in the cells on the surface of the cervix. These abnormal cells can progress to invasive cancer. Once the cancer appears it can progress through four stages. The stages are defined by the extent of spread of the cancer. There are two main types of cervical cancer: (1) squamous type (epidermoid cancer), which may be diagnosed at an early stage by a pap smear; and (2) adenocarcinoma, which is usually detected by a pap smear and pelvic exam. Later stages of cervical cancer cause abnormal vaginal bleeding or a bloodstained discharge at unexpected times, such as between menstrual periods, after intercourse, or after menopause. Abnormal vaginal discharge may be cloudy or bloody or may contain mucus with a bad odor. Advanced stages of the cancer may cause pain. The effects of an IL-29 polypeptide of the present invention designed to treat cervical cancer can be evaluated in a murine cervical cancer model similar to that described in Ahn et al., Hum Gene Ther. 2003 Oct. 10; 14(15):1389-99; Hussain et al., Oncology. 1992; 49(3):237-40; and Sengupta et al., Oncology. 1991; 48(3):258-61.

An IL-29 polypeptide of the present invention can be used to treat head and neck tumors. Most cancers of the head and neck are of a type called carcinoma (in particular squamous cell carcinoma). Carcinomas of the head and neck start in the cells that form the lining of the mouth, nose, throat or ear, or the surface layer covering the tongue. However, cancers of the head and neck can develop from other types of cells. Lymphoma develops from the cells of the lymphatic system. Sarcoma develops from the supportive cells which make up muscles, cartilage or blood vessels. Melanoma starts from cells called melanocytes, which give colour to the eyes and skin. The symptoms of a head and neck cancer will depend on its location—for example, cancer of the tongue may cause some slurring of speech. The most common symptoms are an ulcer or sore area in the head or neck that does not heal within a few weeks; difficulty in swallowing, or pain when chewing or swallowing; trouble with breathing or speaking, such as persistent noisy breathing, slurred speech or a hoarse voice; a numb feeling in the mouth; a persistent blocked nose, or nose bleeds; persistent earache, ringing in the ear, or difficulty in hearing; a swelling or lump in the mouth or neck; pain in the face or upper jaw; in people who smoke or chew tobacco, pre-cancerous changes can occur in the lining of the mouth, or on the tongue. These can appear as persistent white patches (leukoplakia) or red patches (erythroplakia). They are usually painless but can sometimes be sore and may bleed (Cancerbacup Internet website). The effects of an IL-29 polypeptide of the present invention designed for treating head and neck cancers can be evaluated in a murine head and neck tumor model similar to that described in Kuriakose et al., Head Neck. 2000 January; 22(1):57-63; Cao et al., Clin Cancer Res. 1999 July; 5(7):1925-34; Hier et al., Laryngoscope. 1995 October; 105(10):1077-80; Braakhuis et al., Cancer Res. 1991 Jan. 1; 51(1):211-4; Baker, S. R., Laryngoscope. 1985 January; 95(1):43-56; and Dong et al., Cancer Gene Ther. 2003 February; 10(2):96-104.

An IL-29 polypeptide of the present invention can be used to treat brain cancers, such as gliomas. Tumors that begin in brain tissue are known as primary tumors of the brain. Primary brain tumors are named according to the type of cells or the part of the brain in which they begin. The most common primary brain tumors are gliomas, e.g., gliobastoma multiforme and anaplastic astrocytoma. They begin in glial cells. There are many types of gliomas. Astrocytomas arise from star-shaped glial cells called astrocytes. In adults, astrocytomas most often arise in the cerebrum. In children, they occur in the brain stem, the cerebrum, and the cerebellum. A grade III astrocytoma is sometimes called an anaplastic astrocytoma. A grade IV astrocytoma is usually called a glioblastoma multiforme. Brain stem gliomas occur in the lowest part of the brain. Ependymomas arise from cells that line the ventricles or the central canal of the spinal cord. Oligodendrogliomas arise from cells that make the fatty substance that covers and protects nerves. These tumors usually occur in the cerebrum. They grow slowly and usually do not spread into surrounding brain tissue. The symptoms of brain tumors depend on tumor size, type, and location. Symptoms may be caused when a tumor presses on a nerve or damages a certain area of the brain. They also may be caused when the brain swells or fluid builds up within the skull. These are the most common symptoms of brain tumors: Headaches; Nausea or vomiting; Changes in speech, vision, or hearing; Problems balancing or walking; Changes in mood, personality, or ability to concentrate; Problems with memory; Muscle jerking or twitching (seizures or convulsions); and Numbness or tingling in the arms or legs. The effects of an IL-29 polypeptide of the present invention designed to treat brain cancer can be evaluated in a glioma animal model similar to that described in Schueneman et al., Cancer Res. 2003 Jul. 15; 63(14):4009-16; Martinet et al., Eur J Surg Oncol. 2003 May; 29(4):351-7; Bello et al., Clin Cancer Res. 2002 November; 8(11):3539-48; Ishikawa et al., Cancer Sci. 2004 January; 95(1):98-103; Degen et al., J. Neurosurg. 2003 November; 99(5):893-8; Engelhard et al., Neurosurgery. 2001 March; 48(3):616-24; Watanabe et al., Neurol Res. 2002 July; 24(5):485-90; and Lumniczky et al., Cancer Gene Ther. 2002 January; 9(1):44-52.

An IL-29 polypeptide of the present invention can be used to treat thyroid cancer. Papillary and follicular thyroid cancers account for 80 to 90 percent of all thyroid cancers. Both types begin in the follicular cells of the thyroid. Most papillary and follicular thyroid cancers tend to grow slowly. Medullary thyroid cancer accounts for 5 to 10 percent of thyroid cancer cases. Anaplastic thyroid cancer is the least common type of thyroid cancer (only 1 to 2 percent of cases). The cancer cells are highly abnormal and difficult to recognize. This type of cancer is usually very hard to control because the cancer cells tend to grow and spread very quickly. Early thyroid cancer often does not cause symptoms. But as the cancer grows, symptoms may include: A lump, or nodule, in the front of the neck near the prominentia laryngea; Hoarseness or difficulty speaking in a normal voice; Swollen lymph nodes, especially in the neck; Difficulty swallowing or breathing; or Pain in the throat or neck. The effects of an IL-29 polypeptide of the present invention designed for the treatment of thyroid cancer can be evaluated in a murine or rat thyroid tumor model similar to that described in Quidville et al., Endocrinology. 2004 May; 145(5):2561-71 (mouse model); Cranston et al., Cancer Res. 2003 Aug. 15; 63(16):4777-80 (mouse model); Zhang et al., Clin Endocrinol (Oxf). 2000 June; 52(6):687-94 (rat model); and Zhang et al., Endocrinology. 1999 May; 140(5):2152-8 (rat model).

An IL-29 polypeptide of the present invention can be used to treat liver cancer. There are two different types of primary liver cancer. The most common kind is called hepatoma or hepatocellular carcinoma (HCC), and arises from the main cells of the liver (the hepatocytes). This type is usually confined to the liver, although occasionally it spreads to other organs. There is also a rarer sub-type of hepatoma called Fibrolamellar hepatoma. The other type of primary liver cancer is called cholangiocarcinoma or bile duct cancer, because it starts in the cells lining the bile ducts. Most people who develop hepatoma usually also have a condition called cirrhosis of the liver. Infection with either the hepatitis B or hepatitis C virus can lead to liver cancer, and can also be the cause of cirrhosis, which increases the risk of developing hepatoma. People who have a rare condition called haemochromatosis, which causes excess deposits of iron in the body, have a higher chance of developing hepatoma. Thus, an scF c molecule of the present invention may be used to treat, prevent, inhibit the progression of, delay the onset of, and/or reduce the severity or inhibit at least one of the conditions or symptoms associated with hepatocellular carcinoma. The effects of an IL-29 polypeptide of the present invention designed to treat liver cancer can be evaluated in a hepatocellular carcinoma transgenic mouse model, which includes the overexpression of transforming growth factor-.alpha. (TFG-.alpha.) alone (Jhappan et al., Cell, 61:1137-1146 (1990); Sandgren et al., Mol. Cell. Biol., 13:320-330 (1993); Sandgren et al., Oncogene, 4:715-724 (1989); and Lee et al., Cancer Res., 52:5162:5170 (1992)) or in combination with c-myc (Murakami et al., Cancer Res., 53:1719-1723 (1993), mutated H-ras (Saitoh et al., Oncogene, 5:1195-2000 (1990)), hepatitis B viral genes encoding HbsAg and HBx (Toshkov et al., Hepatology, 20:1162-1172 (1994) and Koike et al., Hepatology, 19:810-819 (1994)), SV40 large T antigen (Sepulveda et al., Cancer Res., 49:6108-6117 (1989) and Schirmacher et al., Am. J. Pathol., 139:231-241 (1991)) and FGF19 (Nicholes et al., American Journal of Pathology, 160(6):2295-2307 (June 2002)).

An IL-29 polypeptide of the present invention can be used to treat lung cancer (e.g., small cell lung cancer, non-small cell lung cancer such as Squamous cell carcinoma, Adenocarcinoma and Large cell carcinoma, and mesothelioma). The effects of an IL-29 polypeptide of the present invention designed to treat a lung cancer can be evaluated in a human small/non-small cell lung carcinoma xenograft model. Briefly, human tumors are grafted into immunodecicient mice and these mice are treated with an IL-29 polypeptide of the present invention alone or in combination with other agents which can be used to demonstrate the efficacy of the treatment by evaluating tumor growth (Nemati et al., Clin Cancer Res. 2000 May; 6(5):2075-86; and Hu et al., Clin Cancer Res. 2004 Nov. 15; 10(22):7662-70).

2. Endpoints and Anti-tumor Activity for Solid Tumors

While each protocol may define tumor response assessments differently, the RECIST (Response evaluation Criteria in solid tumors) criteria is currently considered to be the recommended guidelines for assessment of tumor response by the National Cancer Institute (see Therasse et al., J. Natl. Cancer Inst. 92:205-216, 2000). According to the RECIST criteria tumor response means a reduction or elimination of all measurable lesions or metastases. Disease is generally considered measurable if it comprises lesions that can be accurately measured in at least one dimension as >20 mm with conventional techniques or >10 mm with spiral CT scan with clearly defined margins by medical photograph or X-ray, computerized axial tomography (CT), magnetic resonance imaging (MRI), or clinical examination (if lesions are superficial). Non-measurable disease means the disease comprises of lesions <20 mm with conventional techniques or <10 mm with spiral CT scan, and truely non-measurable lesions (too small to accurately measure). Non-measurable disease includes pleural effusions, ascites, and disease documented by indirect evidence.

The criteria for objective status are required for protocols to assess solid tumor response. Representative criteria include the following: (1) Complete Response (CR) defined as complete disappearance of all measurable and evaluable disease. No new lesions. No disease related symptoms. No evidence of non-evaluable disease; (2) Partial Response (PR) defined as greater than or equal to 50% decrease from baseline in the sum of products of perpendicular diameters of all measurable lesions. No progression of evaluable disease. No new lesions. Applies to patients with at least one measurable lesion; (3) Progression defined as 50% or an increase of 10 cm.sup.2 in the sum of products of measurable lesions over the smallest sum observed using same techniques as baseline, or clear worsening of any evaluable disease, or reappearance of any lesion which had disappeared, or appearance of any new lesion, or failure to return for evaluation due to death or deteriorating condition (unless unrelated to this cancer); (4) Stable or No Response defined as not qualifying for CR, PR, or Progression. (See, Clinical Research Associates Manual, ibid.)

Additional endpoints that are accepted within the oncology art include overall survival (OS), disease-free survival (DFS), objective response rate (ORR), time to progression (TTP), and progression-free survival (PFS) (see, Guidance for Industry: Clinical Trial Endpoints for the Approval of Cancer Drugs and Biologics, April 2005, Center for Drug Evaluation and Research, FDA, Rockville, Md.)

### a. Chemotherapy Combinations

In certain embodiments, an IL-29 polypeptide of the present invention is administered in combination with one or more chemotherapeutic agents. Chemotherapeutic agents have different modes of actions, for example, by influencing either DNA or RNA and interfering with cell cycle replication. Examples of chemotherapeutic agents that act at the DNA level or on the RNA level are anti-metabolites (such as Azathioprine, Cytarabine, Fludarabine phosphate, Fludarabine, Gemcitabine, cytarabine, Cladribine, capecitabine 6-mercaptopurine, 6-thioguanine, methotrexate, 5-fluoroouracil and hyroxyurea); alkylating agents (such as Melphalan, Busulfan, Cis-platin, Carboplatin, Cyclophosphamide, Ifosphamide, Dacarabazine, Procarbazine, Chlorambucil, Thiotepa, Lomustine, Temozolamide); anti-mitotic agents (such as Vinorelbine, Vincristine, Vinblastine, Docetaxel, Paclitaxel); topoisomerase inhibitors (such as Doxorubincin, Amsacrine, Irinotecan, Daunorubicin, Epirubicin, Mitomycin, Mitoxantrone, Idarubicin, Teniposide, Etoposide, Topotecan); antibiotics (such as actinomycin and bleomycin); asparaginase; anthracyclines or taxanes.

### b. Radiotherapy Combinations

In some variations, an IL-29 polypeptide of the present invention is administered in combination with radiotherapy. Certain tumors can be treated with radiation or radiopharmaceuticals. Radiation therapy is generally used to treat unresectable or inoperable tumors and/or tumor metastases. Radiotherapy is typically delivered in three ways. External beam irradiation is administered at distance from the body and includes gamma rays (60Co) and X-rays. Brachytherapy uses sources, for example .sup.60Co, .sup.137Cs, .sup.1921r, or .sup.125I, with or in contact with a target tissue.

### c. Hormonal Agent Combinations

In some embodiments, an IL-29 polypeptide of the present invention is administered in combination with a hormone or anti-hormone. Certain cancers are associated with hormonal dependency and include, for example, ovarian cancer, breast cancer, and prostate cancer. Hormonal-dependent cancer treatment may comprise use of anti-androgen or anti-estrogen compounds. Hormones and anti-hormones used in cancer therapy include Estramustine phosphate, Polyestradiol phosphate, Estradiol, Anastrozole, Exemestane, Letrozole, Tamoxifen, Megestrol acetate, Medroxyprogesterone acetate, Octreotide, Cyproterone acetate, Bicaltumide, Flutamide, Tritorelin, Leuprorelin, Buserelin and Goserelin.

C. Inflammation and Autoimmunity Treatment

Diseases of the immune system are significant healthcare problems that are growing at epidemic proportions. As such, they require novel, aggressive approaches to the development of new therapeutic agents. Standard therapy for autoimmune disease has been high dose, long-term systemic corticosteroids and immunosuppressive agents. The drugs used fall into three major categories: (1) glucocorticoids, such as prednisone and prednisolone; (2) calcineurin inhibitors, such as cyclosporine and tacrolimus; and (3) antiproliferative/antimetabolic agents such as azathioprine, sirolimus, and mycophenolate mofetil. Although these drugs have met with high clinical success in treating a number of autoimmune conditions, such therapies require lifelong use and act nonspecifically to suppress the entire immune system. The patients are thus exposed to significantly higher risks of infection and cancer. The calcineurin inhibitors and steroids are also nephrotoxic and diabetogenic, which has limited their clinical utility.

In addition to the conventional therapies for autoimmune disease, monoclonal antibodies and soluble receptors that target cytokines and their receptors have shown efficacy in a variety of autoimmune and inflammation diseases such as rheumatoid arthritis, organ transplantation, and Crohn's disease. Some of the agents include infliximab (REMICADE) and etanercept (ENBREL) that target tumor necrosis factor (TNF), muromonab-CD3 (ORTHOCLONE OKT3) that targets the T cell antigen CD3, and daclizumab (ZENAPAX) that binds to CD25 on activated T cells, inhibiting signaling through this pathway. While efficacious in treating certain inflammatory conditions, use of these drugs has been limited by side effects including the “cytokine release syndrome” and an increased risk of infection.

Passive immunization with intravenous immunoglobulin (IVIG) was licensed in the United States in 1981 for replacement therapy in patients with primary antibody deficiencies. IVIG is obtained from the plasma of large numbers (10,000-20,000) of healthy donors by cold ethanol fractionation. Commonly used IVIG preparations include Sandoglobulin, Flebogamma, Gammagard, Octagam, and Vigam S.

Subsequent investigation showed that IVIG was also effective in ameliorating autoimmune symptoms in Kawasaki's disease and immune thrombocytopenia purpura. IVIG has also been shown to reduce inflammation in adult dermatomyositis, Guillian-Barre syndrome, chronic inflammatory demyelinating polyneuropathies, multiple sclerosis, vasculitis, uveitis, myasthenia gravis, and in the Lambert-Eaton syndrome. Numerous mechanisms have been proposed to explain the mode of action of IVIG, including regulation of Fc gamma receptor expression, increased clearance of pathogenic antibodies due to saturation of the neonatal Fc receptor FcRn, attenuation of complement-mediated damage, and modulation of T and B cells or the reticuloendothelial system. Since Fc domains purified from IVIG are as active as intact IgG in a number of in vitro and in vivo models of inflammation, it is well accepted that the anti-inflammatory properties of IVIG reside in the Fc domain of the IgG. In general, efficacy is seen when only large amounts of IVIG are infused into a patient, with an average dose of 2 g/kg/month used in autoimmune disease.

The common (1-10% of patients) side effects of IVIG treatment include flushing, fever, myalgia, back pain, headache, nausea, vomiting, arthralgia, and dizziness. Uncommon (0.1-1% of patients) side effects include anaphylaxis, aseptic meningitis, acute renal failure, haemolytic anemia, and eczema. Although IVIG is generally considered safe, the pooled human plasma source is considered to be risk factor for transfer of infectious agents. Thus, the use of IVIG is limited by its availability, high cost ($100/gm, including infusion cost), and the potential for severe adverse reactions. Thus, it would be significantly advantageous to develop a therapeutic that offered the efficacy of IVIG without the numerous issues described above (undue side effects and cost/availability issues).

As such, the present invention concerns compositions and methods useful for the diagnosis and treatment of immune related disease in mammals, including humans. The present invention is based on the identification of IL-29 polypeptides which inhibit the immune response in mammals and may be used to treat inflammatory and immune diseases or conditions such as acute or chronic inflammation, ulcerative colitis, chronic bronchitis, asthma, emphysema, myositis, polymyositis, immune dysregulation diseases, cachexia, septicemia, atherosclerosis, psoriasis, psoriatic arthritis, atopic dermatitis, inflammatory skin conditions, rheumatoid arthritis, inflammatory bowel disease (IBD), Crohn's Disease, diverticulosis, pancreatitis, type I diabetes (IDDM), pancreatic cancer, pancreatitis, Graves Disease, colon and intestinal cancer, autoimmune disease, sepsis, organ or bone marrow transplant rejection; inflammation due to endotoxemia, trauma, surgery or infection; amyloidosis; splenomegaly; graft versus host disease; and where inhibition of inflammation, immune suppression, reduction of proliferation of hematopoietic, immune, inflammatory or lymphoid cells, macrophages, T-cells (including Th1 and Th2 cells), suppression of immune response to a pathogen or antigen. Immunotherapy of autoimmune disorders using antibodies which target B-cells is described in PCT Application Publication No. WO 00/74718. Exemplary autoimmune diseases are acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, parnphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, and fibrosing alveolitis.

Inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g., psoriasis, arthritis, rheumatoid arthritis, multiple sclerosis, inflammatory bowel disease and the like), septic shock and multiple organ failure. Importantly, these diverse disease states share common inflammatory mediators. The collective diseases that are characterized by inflammation have a large impact on human morbidity and mortality. Therefore it is clear that an IL-29 polypeptide of the present invention could have crucial therapeutic potential for a vast number of human and animal diseases, from asthma and allergy to autoimmunity and septic shock.

1. Arthritis

Arthritis, including osteoarthritis, rheumatoid arthritis, arthritic joints as a result of injury, and the like, are common inflammatory conditions which would benefit from the therapeutic use of an IL-29 polypeptide of the present invention. For example, rheumatoid arthritis (RA) is a systemic disease that affects the entire body and is one of the most common forms of arthritis. It is characterized by the inflammation of the membrane lining the joint, which causes pain, stiffness, warmth, redness and swelling. Inflammatory cells release enzymes that may digest bone and cartilage. As a result of rheumatoid arthritis, the inflamed joint lining, the synovium, can invade and damage bone and cartilage leading to joint deterioration and severe pain amongst other physiologic effects. The involved joint can lose its shape and alignment, resulting in pain and loss of movement.

Rheumatoid arthritis (RA) is an immune-mediated disease particularly characterized by inflammation and subsequent tissue damage leading to severe disability and increased mortality. A variety of cytokines are produced locally in the rheumatoid joints. Numerous studies have demonstrated that IL-1 and TNF-alpha, two prototypic pro-inflammatory cytokines, play an important role in the mechanisms involved in synovial inflammation and in progressive joint destruction. Indeed, the administration of TNF-alpha and IL-1 inhibitors in patients with RA has led to a dramatic improvement of clinical and biological signs of inflammation and a reduction of radiological signs of bone erosion and cartilage destruction. However, despite these encouraging results, a significant percentage of patients do not respond to these agents, suggesting that other mediators are also involved in the pathophysiology of arthritis (Gabay, Expert. Opin. Biol. Ther. 2(2):135-149, 2002).

There are several animal models for rheumatoid arthritis known in the art. For example, in the collagen-induced arthritis (CIA) model, mice develop chronic inflammatory arthritis that closely resembles human rheumatoid arthritis. Since CIA shares similar immunological and pathological features with RA, this makes it an ideal model for screening potential human anti-inflammatory compounds. The CIA model is a well-known model in mice that depends on both an immune response, and an inflammatory response, in order to occur. The immune response comprises the interaction of B-cells and CD4+ T-cells in response to collagen, which is given as antigen, and leads to the production of anti-collagen antibodies. The inflammatory phase is the result of tissue responses from mediators of inflammation, as a consequence of some of these antibodies cross-reacting to the mouse's native collagen and activating the complement cascade. An advantage in using the CIA model is that the basic mechanisms of pathogenesis are known. The relevant T-cell and B-cell epitopes on type II collagen have been identified, and various immunological (e.g., delayed-type hypersensitivity and anti-collagen antibody) and inflammatory (e.g., cytokines, chemokines, and matrix-degrading enzymes) parameters relating to immune-mediated arthritis have been determined, and can thus be used to assess test compound efficacy in the CIA model (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995).

The administration of an IL-29 polypeptide of the present invention to these CIA model mice is used to evaluate the use of such molecules as a therapeutic useful in ameliorating symptoms and altering the course of disease. By way of example and without limitation, the injection of 10-200.micro.g of such an antibody fragment of the present invention per mouse (one to seven times a week for up to but not limited to 4 weeks via s.c., i.p., or i.m route of administration) can significantly reduce the disease score (paw score, incidence of inflammation, or disease). Depending on the initiation of administration (e.g. prior to or at the time of collagen immunization, or at any time point following the second collagen immunization, including those time points at which the disease has already progressed), such antibody fragments can be efficacious in preventing rheumatoid arthritis, as well as preventing its progression.

2. Endotoxemia

Endotoxemia is a severe condition commonly resulting from infectious agents such as bacteria and other infectious disease agents, sepsis, toxic shock syndrome, or in immunocompromised patients subjected to opportunistic infections, and the like. Therapeutically useful of anti-inflammatory proteins, such as antibodies of the invention, could aid in preventing and treating endotoxemia in humans and animals. Such IL-29 polypeptides of the present invention could serve as a valuable therapeutic to reduce inflammation and pathological effects in endotoxemia.

Lipopolysaccharide (LPS) induced endotoxemia engages many of the proinflammatory mediators that produce pathological effects in the infectious diseases and LPS induced endotoxemia in rodents is a widely used and acceptable model for studying the pharmacological effects of potential pro-inflammatory or immunomodulating agents. LPS, produced in gram-negative bacteria, is a major causative agent in the pathogenesis of septic shock (Glausner et al., Lancet 338:732, 1991). A shock-like state can indeed be induced experimentally by a single injection of LPS into animals. Molecules produced by cells responding to LPS can target pathogens directly or indirectly. Although these biological responses protect the host against invading pathogens, they may also cause harm. Thus, massive stimulation of innate immunity, occurring as a result of severe Gram-negative bacterial infection, leads to excess production of cytokines and other molecules, and the development of a fatal syndrome, septic shock syndrome, which is characterized by fever, hypotension, disseminated intravascular coagulation, and multiple organ failure (Dumitru et al. Cell 103:1071-1083, 2000).

These toxic effects of LPS are mostly related to macrophage activation leading to the release of multiple inflammatory mediators. Among these mediators, TNF appears to play a crucial role, as indicated by the prevention of LPS toxicity by the administration of neutralizing anti-TNF antibodies (Beutler et al., Science 229:869, 1985). It is well established that 1.micro.g injection of E. coli LPS into a C57B1/6 mouse will result in significant increases in circulating IL-6, TNF-alpha, IL-1, and acute phase proteins (for example, SAA) approximately 2 hours post injection. The toxicity of LPS appears to be mediated by these cytokines as passive immunization against these mediators can result in decreased mortality (Beutler et al., Science 229:869, 1985). The potential immunointervention strategies for the prevention and/or treatment of septic shock include anti-TNF mAb, IL-1 receptor antagonist, LIF, IL-10, and G-CSF.

The administration of an IL-29 polypeptide of the present invention to an LPS-induced model may be used to evaluate the use of such antibody fragments to ameliorate symptoms and alter the course of LPS-induced disease. Moreover, results showing inhibition of immune response by such antibody fragments of the invention provide proof of concept that such IL-29 polypeptides can also be used to ameliorate symptoms in the LPS-induced model and alter the course of disease. The model will show induction of disease specific cytokines by LPS injection and the potential treatment of disease by such antibody fragments. Since LPS induces the production of pro-inflammatory factors possibly contributing to the pathology of endotoxemia, the neutralization of pro-inflammatory factors by an IL-29 polypeptide of the present invention can be used to reduce the symptoms of endotoxemia, such as seen in endotoxic shock.

3. Inflammatory Bowel Disease IBD

In the United States approximately 500,000 people suffer from Inflammatory Bowel Disease (IBD) which can affect either colon and rectum (Ulcerative colitis) or both, small and large intestine (Crohn's Disease). The pathogenesis of these diseases is unclear, but they involve chronic inflammation of the affected tissues. An IL-29 polypeptide of the present invention could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress.

Although there is currently no cure for UC available, treatments are focused on suppressing the abnormal inflammatory process in the colon lining. Treatments including corticosteroids, immunosuppressives (e.g. azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanied by the clinical picture of progressive weight loss (wasting), bloody diarrhea, rectal prolapse, and large bowel wall thickening (Neurath et al. Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of an IL-29 polypeptide of the present invention to these TNBS or DSS models can be used to evaluate the use such antibody fragments to ameliorate symptoms and alter the course of gastrointestinal disease.

4. Psoriasis

Psoriasis is a chronic skin condition that affects more than seven million Americans. Psoriasis occurs when new skin cells grow abnormally, resulting in inflamed, swollen, and scaly patches of skin where the old skin has not shed quickly enough. Plaque psoriasis, the most common form, is characterized by inflamed patches of skin (“lesions”) topped with silvery white scales. Psoriasis may be limited to a few plaques or involve moderate to extensive areas of skin, appearing most commonly on the scalp, knees, elbows and trunk. Although it is highly visible, psoriasis is not a contagious disease. The pathogenesis of the diseases involves chronic inflammation of the affected tissues. The IL-29 polypeptides of the present invention could serve as a valuable therapeutic to reduce inflammation and pathological effects in psoriasis, other inflammatory skin diseases, skin and mucosal allergies, and related diseases.

Psoriasis is a T-cell mediated inflammatory disorder of the skin that can cause considerable discomfort. It is a disease for which there is no cure and affects people of all ages. Psoriasis affects approximately two percent of the populations of European and North America. Although individuals with mild psoriasis can often control their disease with topical agents, more than one million patients worldwide require ultraviolet or systemic immunosuppressive therapy. Unfortunately, the inconvenience and risks of ultraviolet radiation and the toxicities of many therapies limit their long-term use. Moreover, patients usually have recurrence of psoriasis, and in some cases rebound, shortly after stopping immunosuppressive therapy.

In addition to other disease models described herein, the activity of antibody fragments of the invention on inflammatory tissue derived from human psoriatic lesions can be measured in vivo using a severe combined immune deficient (SCID) mouse model. Several mouse models have been developed in which human cells are implanted into immunodeficient mice (collectively referred to as xenograft models); see, for example, Cattan A R, Douglas E, Leuk. Res. 18:513-22, 1994 and Flavell, D J, Hematological Oncology 14:67-82, 1996. As an in vivo xenograft model for psoriasis, human psoriatic skin tissue is implanted into the SCID mouse model, and challenged with an appropriate antagonist. Moreover, other psoriasis animal models in ther art may be used to evaluate the IL-29 polypeptides of the present invention, such as human psoriatic skin grafts implanted into AGR129 mouse model, and challenged with an appropriate antagonist (e.g., see, Boyman, O. et al., J. Exp. Med. Online publication #20031482, 2004, incorporated herein by reference). Similarly, tissues or cells derived from human colitis, IBD, arthritis, or other inflammatory lesions can be used in the SCID model to assess the anti-inflammatory properties of the antibody fragments of the invention described herein.

Therapies designed to abolish, retard, or reduce inflammation using antibody fragments of the invention can be tested by administration of such antibodies to SCID mice bearing human inflammatory tissue (e.g., psoriatic lesions and the like), or other models described herein. Efficacy of treatment is measured and statistically evaluated as increased anti-inflammatory effect within the treated population over time using methods well known in the art. Some exemplary methods include, but are not limited to measuring for example, in a psoriasis model, epidermal thickness, the number of inflammatory cells in the upper dermis, and the grades of parakeratosis. Such methods are known in the art and described herein. For example, see Zeigler, M. et al. Lab Invest 81:1253, 2001; Zollner, T. M. et al. J. Clin. Invest. 109:671, 2002; Yamanaka, N. et al. Microbio.l Immunol. 45:507, 2001; Raychaudhuri, S. P. et al. Br. J. Dermatol. 144:931, 2001; Boehncke, W. H et al. Arch. Dermatol. Res. 291:104, 1999; Boehncke, W. H et al. J. Invest. Dermatol. 116:596, 2001; Nickoloff, B. J. et al. Am. J. Pathol. 146:580, 1995; Boehncke, W. H et al. J. Cutan. Pathol. 24:1, 1997; Sugai, J., M. et al. J. Dermatol. Sci. 17:85, 1998; and Villadsen L. S. et al. J. Clin. Invest. 112:1571, 2003. Inflammation may also be monitored over time using well-known methods such as flow cytometry (or PCR) to quantitate the number of inflammatory or lesional cells present in a sample, score (weight loss, diarrhea, rectal bleeding, colon length) for IBD, paw disease score and inflammation score for CIA RA model.

Moreover, psoriasis is a chronic inflammatory skin disease that is associated with hyperplastic epidermal keratinocytes and infiltrating mononuclear cells, including CD4+ memory T cells, neutrophils and macrophages (Christophers, Int. Arch. Allergy Immunol., 110:199, 1996). It is currently believed that environmental antigens play a significant role in initiating and contributing to the pathology of the disease. However, it is the loss of tolerance to self-antigens that is thought to mediate the pathology of psoriasis. Dendritic cells and CD4+ T cells are thought to play an important role in antigen presentation and recognition that mediate the immune response leading to the pathology. We have recently developed a model of psoriasis based on the CD4+CD45RB transfer model (Davenport et al., Internat. Immunopharmacol., 2:653-672). The IL-29 polypeptides of the present invention are administered to the mice Inhibition of disease scores (skin lesions, inflammatory cytokines) indicates the effectiveness of such antibodies in psoriasis.

5. Atopic Dermatitis.

AD is a common chronic inflammatory disease that is characterized by hyperactivated cytokines of the helper T cell subset 2 (Th2). Although the exact etiology of AD is unknown, multiple factors have been implicated, including hyperactive Th2 immune responses, autoimmunity, infection, allergens, and genetic predisposition. Key features of the disease include xerosis (dryness of the skin), pruritus (itchiness of the skin), conjunctivitis, inflammatory skin lesions, Staphylococcus aureus infection, elevated blood eosinophilia, elevation of serum IgE and IgG1, and chronic dermatitis with T cell, mast cell, macrophage and eosinophil infiltration. Colonization or infection with S. aureus has been recognized to exacerbate AD and perpetuate chronicity of this skin disease.

AD is often found in patients with asthma and allergic rhinitis, and is frequently the initial manifestation of allergic disease. About 20% of the population in Western Countries suffers from these allergic diseases, and the incidence of AD in developed countries is rising for unknown reasons. AD typically begins in childhood and can often persist through adolescence into adulthood. Current treatments for AD include topical corticosteroids, oral cyclosporin A, non-corticosteroid immunosuppressants such as tacrolimus (FK506 in ointment form), and interferon-gamma. Despite the variety of treatments for AD, many patients' symptoms do not improve, or they have adverse reactions to medications, requiring the search for other, more effective therapeutic agents.

Pharmaceutical Compositions or Compositions. For pharmaceutical use, an IL-29 polypeptide of the present invention is formulated as a pharmaceutical composition. A pharmaceutical composition comprising an IL-29 polypeptide of the present invention can be formulated according to known methods for preparing pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. In one embodiment, the IL-29 polypeptides of the present invention are formulated for parenteral, particularly intravenous or subcutaneous, delivery according to conventional methods. Intravenous administration will be by bolus injection, controlled release, e.g., using mini-pumps or other appropriate technology, or by infusion over a typical period of one to several hours. In general, pharmaceutical formulations will include an IL-29 polypeptide of the present invention in combination with a pharmaceutically acceptable carrier, such as saline, buffered saline, 5% dextrose in water or the like. Formulations may further include one or more excipients, preservatives, solubilizers, buffering agents, albumin to prevent protein loss on vial surfaces, etc. When utilizing such a combination therapy, the IL-29 polypeptides of the present invention may be combined in a single formulation or may be administered in separate formulations. Methods of formulation are well known in the art and are disclosed, for example, in Remington's Pharmaceutical Sciences, Gennaro, ed., Mack Publishing Co., Easton Pa., 1990, which is incorporated herein by reference. Therapeutic doses will generally be in the range of 0.1 to 100 mg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. Monospecific antagonists can be individually formulated or provided in a combined formulation. The IL-29 polypeptides of the present invention can also be administered in combination with other cytokines such as IL-3, -6 and -11; stem cell factor; erythropoietin; G-CSF and GM-CSF.

A composition comprising an IL-29 polypeptide of the present invention is administered to a patient in an effective amount. Generally, the dosage of administered an IL-29 polypeptide of the present invention will vary depending upon such factors as the patient's age, weight, height, sex, general medical condition and previous medical history. Typically, it is desirable to provide the recipient with a dosage which is in the range of from about 1 pg/kg to 10 mg/kg (amount of agent/body weight of patient), although a lower or higher dosage also may be administered as circumstances dictate.

Administration of an IL-29 polypeptide of the present invention to a patient can be intravenous, intraarterial, intraperitoneal, intramuscular, subcutaneous, intrapleural, intrathecal, by perfusion through a regional catheter, or by direct intralesional injection. For prevention and treatment purposes, an antagonist may be administered to a patient in a single bolus delivery, via continuous delivery (e.g., continuous transdermal delivery) over an extended time period, or in a repeated administration protocol (e.g., on an hourly, daily, or weekly basis). When administering therapeutic proteins by injection, the administration may be by continuous infusion or by single or multiple boluses. For pharmaceutical use for treatment of neovascular ocular disorders, the IL-29 polypeptides are typically formulated for intravitreal injection according to conventional methods.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising an IL-29 polypeptide of the present invention can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer the IL-29 polypeptide.

A composition comprising an IL-29 polypeptide of the present invention can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, an IL-29 polypeptide of the present invention and a pharmaceutically acceptable carrier are administered to a patient in a therapeutically effective amount. A combination of a therapeutic IL-29 polypeptide of the present invention invention and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response. Effective treatment may be assessed in a variety of ways. In one embodiment, effective treatment is determined by reduced inflammation. In other embodiments, effective treatment is marked by inhibition of inflammation. In still other embodiments, effective therapy is measured by increased well-being of the patient including such signs as weight gain, regained strength, decreased pain, thriving, and subjective indications from the patient of better health.

Determination of effective dosages in this context is typically based on animal model studies followed up by human clinical trials and is guided by determining effective dosages and administration protocols that significantly reduce the occurrence or severity of the patient disease or disorder in model subjects. Effective doses of the compositions of the present invention vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, e.g., to optimize safety and efficacy. Accordingly, a therapeutically or prophylactically effective amount is also one in which any undesired collateral effects are outweighed by beneficial effects of inhibiting angiogenesis. For example, administration of an IL-29 polypeptide of the present invention may have a dosage range from about 0.1.micro.g to 100 mg/kg or 1.micro.g/kg to about 50 mg/kg, and more usually 10.micro.g to 5 mg/kg of the patient's body weight. In more specific embodiments, an effective amount of the agent is between about 1.micro.g/kg and about 20 mg/kg, between about 10.micro.g/kg and about 10 mg/kg, or between about 0.1 mg/kg and about 5 mg/kg. Dosages within these ranges can be achieved by single or multiple administrations, including, e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bi-monthly intervals. Alternatively, administrations can be on an irregular basis as indicated by monitoring of a marker such as NK cell activity and/or clinical symptoms of the disease or disorder.

Dosage of the pharmaceutical composition may be varied by the attending clinician to maintain a desired concentration at a target site. For example, if an intravenous mode of delivery is selected, local concentration of the agent in the bloodstream at the target tissue may be between about 1-50 nanomoles of the composition per liter, sometimes between about 1.0 nanomole per liter and 10, 15, or 25 nanomoles per liter depending on the patient's status and projected measured response. Higher or lower concentrations may be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

A composition comprising an IL-29 polypeptide of the present invention can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants. (See, e.g., Bremer et al., Pharm. Biotechnol. 10:239, 1997; Ranade, “Implants in Drug Delivery,” in Drug Delivery Systems 95-123 (Ranade and Hollinger, eds., CRC Press 1995); Bremer et al., “Protein Delivery with Infusion Pumps,” in Protein Delivery: Physical Systems 239-254 (Sanders and Hendren, eds., Plenum Press 1997); Yewey et al., “Delivery of Proteins from a Controlled Release Injectable Implant,” in Protein Delivery: Physical Systems 93-117 (Sanders and Hendren, eds., Plenum Press 1997).) Other solid forms include creams, pastes, other topological applications, and the like.

Liposomes provide one means to deliver therapeutic polypeptides to a patient intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):561 (1993), Kim, Drugs 46:618 (1993), and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02.micro.m to greater than 10.micro.m. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368 (1985)). After intravenous administration, small liposomes (0.1 to 1.0.micro.m) are typically taken up by cells of the reticuloendothelial system, located principally in the liver and spleen, whereas liposomes larger than 3.0.micro.m are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428 (1984)). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133 (1991); Allen et al., Biochim. Biophys. Acta 1150:9 (1993)).

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960 (1993)). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881 (1997)).

Alternatively, various targeting counter-receptors can be bound to the surface of the liposome, such as antibodies, antibody fragments, carbohydrates, vitamins, and transport proteins. For example, for targeting to the liver, liposomes can be modified with branched type galactosyllipid derivatives to target asialoglycoprotein (galactose) receptors, which are exclusively expressed on the surface of liver cells. (See Kato and Sugiyama, Crit. Rev. Ther. Drug Carrier Syst. 14:287, 1997; Murahashi et al., Biol. Pharm. Bul1.20:259, 1997.) In a more general approach to tissue targeting, target cells are prelabeled with biotinylated antibodies specific for a counter-receptor expressed by the target cell. (See Harasym et al., Adv. Drug Deliv. Rev. 32:99, 1998.) After plasma elimination of free antibody, streptavidin-conjugated liposomes are administered. In another approach, IL-29 polypeptides of the present invention are directly attached to liposomes. (See Harasym et al., supra.)

IL-29 polypeptides of the present invention can be encapsulated within liposomes using standard techniques of protein microencapsulation (see, for example, Anderson et al., Infect. Immun. 31:1099 (1981), Anderson et al., Cancer Res. 50:1853 (1990), and Cohen et al., Biochim. Biophys. Acta 1063:95 (1991), Alving et al. “Preparation and Use of Liposomes in Immunological Studies,” in Liposome Technology, 2nd Edition, Vol. III, Gregoriadis (ed.), page 317 (CRC Press 1993), Wassef et al., Meth. Enzymol. 149:124 (1987)). As noted above, therapeutically useful liposomes may contain a variety of components. For example, liposomes may comprise lipid derivatives of poly(ethylene glycol) (Allen et al., Biochim. Biophys. Acta 1150:9 (1993)).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332 (1995); Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161 (1998); Putney and Burke, Nature Biotechnology 16:153 (1998); Putney, Curr. Opin. Chem. Biol. 2:548 (1998)). Polyethylene glycol (PEG)-coated nanospheres can also provide carriers for intravenous administration of therapeutic proteins (see, for example, Gref et al., Pharm. Biotechnol. 10:167 (1997)).

Other dosage forms can be devised by those skilled in the art, as shown, for example, by Ansel and Popovich, Pharmaceutical Dosage Forms and Drug Delivery Systems, 5th Edition (Lea & Febiger 1990), Gennaro (ed.), Remington's Pharmaceutical Sciences, 19th Edition (Mack Publishing Company 1995), and by Ranade and Hollinger, Drug Delivery Systems (CRC Press 1996).

As an illustration, pharmaceutical compositions may be supplied as a kit comprising a container that comprises an IL-29 polypeptide of the present invention. The IL-29 polypeptides of the present invention can be provided in the form of an injectable solution for single or multiple doses, or as a sterile powder that will be reconstituted before injection. Alternatively, such a kit can include a dry-powder disperser, liquid aerosol generator, or nebulizer for administration of an IL-29 polypeptide of the present invention. Such a kit may further comprise written information on indications and usage of the pharmaceutical composition.

A composition comprising an IL-29 polypeptide of the present invention can be furnished in liquid form, in an aerosol, or in solid form. Liquid forms, are illustrated by injectable solutions, aerosols, droplets, topological solutions and oral suspensions. Solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery Physical Systems, Sanders and Hendren (eds.), pages 93-117 (Plenum Press 1997)). Other solid forms include creams, pastes, other topological applications, and the like.

The present invention provides for compositions comprising IL-29 polypeptides of the present invention that are either administered alone as a therapeutic, or in combination with other agents which are used to treat said condition, as well as methods for and therapeutic uses of the IL-29 polypeptides of the present invention itself. Such compositions can further comprise a pharmaceutical acceptable carrier. The pharmaceutical acceptable carrier can be a conventional organic or inorganic carrier. Examples of carriers include water, buffer solution, alcohol, propylene glycol, macrogol, sesame oil, corn oil, and the like.

The invention is further illustrated by the following non-limiting example.

### EXAMPLES

### Example 1

### Production of IL-29 Mutant Polypeptides

A. Expression Vectors and Strain

The expression vectors used for production of the new IL-29 mutants or variants were generated based on the E. coli ZGOLD5 vector pTAP440. The generation of the construct (pTAP440) used for the production of the polypeptide sequence of SEQ ID NO:14, and its various vector components is described in WO 07/041,713 and U.S. Publication No. 2008-0096252, which is hereby incorporated by reference. In addition, WO 07/041,713 and U.S. Publication No. 2008-0096252 also teach one of skill in the art how to produce IL-29 polypeptides in E. coli, such as ZGOLD5, recover, purify, concentrate and pegylate said IL-29 polypeptides; WO 07/041,713 and U.S. Publication No. 2008-0096252 are hereby incorporated by reference for such purposes. An overview of the plasmids and strains is provided below in Table 6.

TABLE 6
Overview of expression vectors and molecule constructs and
(E. coli ZGOLD5 strain with transfected vector).

| Plasmid | Sequence Identifier | Change relative to pTAP440 |
|---|---|---|
| pTAP440 | SEQ ID NO: 14 | No change |
| | (polypeptide | |
| | sequence of | |
| | pTAP440) | |
| pTAP554 | SEQ ID NO: 6 | IL-29 C1 - deletion of |
| | | carboxy-terminal 14 amino |
| | | acids |
| pTAP555 | SEQ ID NO: 8 | IL-29 C2 - deletion of |
| | | carboxy-terminal 18 amino |
| | | acids |
| pTAP557 | SEQ ID NO: 4 | IL-29 N1 - deletion of amino- |
| | | terminal 12 amino acids, C94S |
| pTAP558 | SEQ ID NO: 10 | IL-29 N1C1 - combination of |
| | | C1 and N1 deletions |
| pTAP559 | SEQ ID NO: 12 | IL-29 N1C2 - combination of |
| | | C2 and N1 deletions |

B. Results

1. Expression and Purification Data

The results from expression and purification of the various constructs are summarized below in Table 7. The data indicates that the original molecule was expressed, recovered, refolded and captured at the shake flask scale. Table 7 is a summary of key process data for the various mutants.

TABLE 7

| | Shake | Solute | | Capture |
|---|---|---|---|---|
| | flask | concentrations | | pool |
| | expression | (g/L of | End of refold | concen- |
| | level | fermentation | concentrations | trations |
| Molecule | (g/L) | broth) | (g/L) | (g/L) |
| IL-29 d2/7 | 0.63 | 5.9 | 0.32 | 0.93 |
| (SEQ ID NO: 14) | | | | |
| IL-29 C1 | 0.29 | 3.1 | 0.12 | 0.34 |
| (SEQ ID NO: 6) | | | | |
| IL-29 C2 | 0.44 | 3.9 | Below limit of | 0.08 |
| (SEQ ID NO: 8) | | | quantitation | |
| IL-29 N1 | 0.35 | 4.0 | 0.18 | 0.55 |
| (SEQ ID NO: 4) | | | | |
| IL-29 N1C1 | 0.69 | 6.1 | 0.17 | 0.50 |
| (SEQ ID NO: 10) | | | | |
| IL-29 N1C2 | 0.47 | 4.1 | Below limit of | N/A |
| (SEQ ID NO: 12) | | | quantitation | |

B. Expression, Refolding and Partial Purification of IL-29 Mutants

The IL-29 mutants were expressed in E. coli using shake flasks as described in WO 07/041,713 and U.S. Publication No. 2008-0096252. Briefly, the expression strains were grown in shake flasks and induced for expression of protein using IPTG. After completion of the fermentation, the fermentation broth was diluted 1:1 with water and the harvested cells were broken open by homogenization. This leads to the release of insoluble inclusion bodies containing recombinant IL-29. Released inclusion bodies were recovered by centrifugation and were subsequently solubilized in guanidine hydrochloride and DTT. This leads to solubilization of IL-29 molecules in a reduced and denatured form. The reduced and denatured IL-29 molecules were refolded using a redox buffer system and captured using cation exchange chromatography.

Two separate data sets were generated for the expression, recovery and refolding of rIL-29.

1. RP-HPLC-TOF Analysis

Approximately 1 μg of sample was injected onto an Ace 3 C-8 300 150×0.5 mm column in 10% acetonitrile/water/0.1% TFA (v/v), resolved by a gradient of 30-70% 10% water/acetonitrile/0.1% TFA (v/v) over 25 minutes, and the wavelength at 280 nm monitored. Masses of proteins were reconstructed from a method developed for analysis of cytokines (ProteinD.m). Briefly, spectra were averaged across the peak, deconvoluted to obtain the measured mass of the protein, and error calculated by comparison of measured to the expected masses of the protein.

The IL-29 mutants were analyzed by RP-HPLC for content. Because expression and refolding levels were low for some of the mutants, IL-29 content had to be calculated outside the standard curve range. Due to the changes in molecule sequence the retention times had to be estimated for quantitative purposes. However, the retention time relative to the control molecule (SEQ ID NO:14) was verified by the RP-HPLC chromatogram retention times in the RP-HPLC-TOF analysis where identity of peaks could be ascribed based on mass determination.

Data from the RP-HPLC-TOF Mass Spectrometry analysis are summarized below in Table 8. The data confirms the sequences for all constructs including oxidized cysteines were as expected based on theoretical mass calculation. Table 8 is a summary of mass spectrometry data from the analysis of capture material for the various constructs from the second set of experiments.

TABLE 8

| | Calculated masses | Observed | | Retention |
|---|---|---|---|---|
| | (Da) including | masses | Error | time |
| IL-29 polypeptide | disulfides | (Da) | (ppm) | (minutes) |
| IL-29 d2/7 | 19590.58 | 19590.48 | 5.10 | 18.54 |
| (SEQ ID NO: 14) | | | | |
| IL-29 C1 | 18108.99 | 18109.21 | 12.14 | 19.07 |
| (SEQ ID NO: 6) | | | | |
| IL-29 C2 | 17660.52 | 17660.29 | 13.02 | 18.81 |
| (SEQ ID NO: 8) | | | | |
| IL-29 N1 | 18395.15 | 18394.46 | 37.51 | 21.42 |
| (SEQ ID NO: 4) | | | | |
| IL-29 N1C1 | 16913.56 | 16913.75 | 11.23 | 21.91 |
| (SEQ ID NO: 10) | | | | |
| IL-29 N1C2 | 16465.09 | 16465.09 | 0.0 | 21.98 |
| (SEQ ID NO: 12) | | | | |

2. RP-HPLC Analysis

Content analysis of the capture pools were performed using reverse phase high pressure liquid chromatography (RP-HPLC). Briefly, samples were analyzed for IL-29 content relative to a IL-29 reference standard (SEQ ID NO:14) using 280 nm wavelength for detection and quantification. Because there was no significant changes in the aromatic amino acid residue content contributing to optical density at this wavelength between the mutants, these numbers were considered accurate for the purposes of calculating yields and relative bioactivity for this set of experiments.

The RP-HPLC content data was used to determine the appropriate dilution levels for samples in the bioassay and to calculate the relative potency of the mutants. The relative retention times suggest that the appropriate peaks were used for quantitation. The retention times relative to the retention times in the RP-HPLC-TOF MS analysis match up. The RP-HPLC retention times from analysis of the capture pools from the second set of experiments is summarized below in Table 9.

TABLE 9

| | Retention time |
|---|---|
| IL-29 Polypeptide | (minutes) |
| IL-29 d2/7 | 4.15 |
| (SEQ ID NO: 14) | |
| IL-29 C1 | 4.39 |
| (SEQ ID NO: 6) | |
| IL-29 C2 | 4.25 |
| (SEQ ID NO: 8) | |
| IL-29 N1 | 5.48 |
| (SEQ ID NO: 4) | |
| IL-29 N1C1 | 5.68 |
| (SEQ ID NO: 10) | |
| IL-29 N1C2 | 5.67 |
| (SEQ ID NO: 12) | |

### Example 2

### Use of IL-29 Mutant Polypeptides in BioAssay

Pool fractions for each refold obtained from the SP550 chromatography were assayed for bioactivity using a cell based potency bioassay. Specifically, the bioassay measures bioactivity using a cell-based assay. The bioassay utilizes a 293 human embryonic kidney (293 HEK) reporter cell line that was engineered to over-express the human IL-29 receptor, and contains a firefly luciferase reporter construct (KZ157), which includes IFN-stimulated response element (ISRE) and signal transducer and activator of transcription (STAT) binding elements placed directly upstream of the luciferase gene. The IL-29 receptor is a heterodimer consisting of IL-10 receptor β (IL-10Rβ) and IL-28 receptor a (IL-28Rα) subunits. Over-expression of the IL-29 receptor was achieved by stable transfection of 293 HEK cells with the IL-28Rα cDNA, which, along with endogenously expressed IL-10Rβ, form the heterodimeric IL-29 receptor. Binding of IL-29 to the IL-29 receptor activates the Janus kinase (Jak)/STAT signaling pathway and results in the formation of the intracellular transcription factor (ISGF3). Subsequent binding of ISGF3 to ISRE/STAT DNA sequence elements results in expression of the firefly luciferase gene product. In the bioassay, the assay cells are stimulated with IL-29 for a defined period of time and then lysed. After addition of a luciferase substrate luciferin to the lysed cells, luciferase expression was measured indirectly in relative light units (RLU) using a luminometer. A calibration curve was generated using an rIL-29 reference standard (SEQ ID NO:14), relating the luminescence signal to the concentration of the IL-29 reference standard, from which the potency of control and test samples were calculated. Results are reported as relative potency units per milligram (RPU/mg), calculated relative to the reference standard. A development reference lot for IL-29 (lot A1408F) was assigned a relative potency unit of 1 per milligram of protein (1 RPU/mg).

Bioactivity data was generated and is summarized below in Table 10. The data shows the average data for the two sets of samples analyzed. The activity of the C1 deletion mutant was comparable to the activity for the original construct (SEQ ID NO:14). None of the other mutants retained bioactivity.

TABLE 10

| | Average specific activity |
|---|---|
| | (RPU/mg) ± deviation from |
| IL-29 Polypeptide | mean |
| IL-29 d2/7 | 0.69 ± 0.02 |
| (SEQ ID NO: 14) | |
| IL-29 C1 | 0.62 ± 0.15 |
| (SEQ ID NO: 6) | |
| IL-29 C2 | Below level of detection |
| (SEQ ID NO: 8) | |
| IL-29 N1 | Below level of detection |
| (SEQ ID NO: 4) | |
| IL-29 N1C1 | Below level of detection |
| (SEQ ID NO: 10 | |
| IL-29 N1C2 | Below level of detection |
| (SEQ ID NO: 12) | |

The full length sequence of IL-29 is not required for the cytokine to retain its biological activity. Specifically, in addition to 6 N-terminal amino acids already eliminated relative to the putative full length mature human sequence (SEQ ID NO:14), an additional 14 amino acids could be deleted from the C-terminal end of the molecule without affecting the molecule's ability to refold and its bioactivity (SEQ ID NO:6). Further deletions at the N- and C-terminal resulted in loss of bioactivity. Overall, the data suggest that a core sequence of IL-29 has been defined that leads to correct three dimensional folding of the molecule, and that this sequence contains all the necessary elements for ligand-receptor interactions required for signaling.

### Example 3

### IL-29 have Antiviral Activity Against Hepatitis B Virus (HBV) In Vivo

A transgenic mouse model (Guidotti et al., J. Virology 69:6158-6169, 1995) supports the replication of high levels of infectious HBV and has been used as a chemotherapeutic model for HBV infection. Transgenic mice are treated with antiviral drugs and the levels of HBV DNA and RNA are measured in the transgenic mouse liver and serum following treatment. HBV protein levels can also be measured in the transgenic mouse serum following treatment. This model has been used to evaluate the effectiveness of lamivudine and IFN-α in reducing HBV viral titers.

HBV TG mice (male) are given intraperitoneal injections of 2.5, 25 or 250 micrograms mouse IL-28 or IL-29 (IL-29 always refers to human IL-29 as there is no mouse IL-29, only mouse IL-28) every other day for 14 days (total of 8 doses). Mice are bled for serum collection on day of treatment (day 0) and day 7. One hour following the final dose of IL-29 mice undergo a terminal bleed and are sacrificed. Serum and liver are analyzed for liver HBV DNA, liver HBV RNA, serum HBV DNA, liver HBc, serum Hbe and serum HBs.

Reduction in liver HBV DNA, liver HBV RNA, serum HBV DNA, liver HBc, serum Hbe or serum HBs in response to IL-29 reflects antiviral activity of these compounds against HBV.

### Example 4

### IL-29 Antiviral Activity Against Hepatitis C Virus in the Hepatitis C Replicon Model

In order to determine the effectiveness of IL-29 or Pegylated IL-29 in preventing viral replication of human hepatitis C virus, the IL-29 or Pegylated IL-29 can be ested in the HCV Replicon model.

In this model AVA5 cells (Huh7 cells containing the subgenomic HCV replicon, BB7) (Blight et al., Science, 290:1972-1974 (2000)) can be used. Cultures are maintained in a sub-confluent state in DMEM with glutamine, non-essential amino acids, and 10% heat-inactivated fetal bovine serum (Biofluids, Inc.) as previously described (Blight et al., Science, 290:1972-1974 (2000)). Stock cultures are maintained in a sub-confluent state in this culture medium with 1 mg/ml G418 (Invitrogen, Inc.) (Blight et al., Science, 290:1972-1974 (2000)). Cells for antiviral analysis are seeded into 24-well or 48-well tissue culture plates (Nunc, Inc.) and are grown for three days in the presence of G418. G418 is then removed for the duration of the antiviral treatments to eliminate potential loss of cells due to the reduction of HCV replicon (and G418-resistance) copy number. Cultures (rapidly dividing, 3-4 cultures per concentration, per experiment) are treated for three consecutive days with the test compounds. Medium is replaced daily with fresh test compounds. Analysis of HCV RNA is performed 24 hours following the last addition of test compounds. Toxicity analyses using neutral red dye uptake are performed as previously described in Korba et al., Antiviral Res., 19(1):55-70 (1992). Cultures for the toxicity analyses are seeded from the same stock cultures and maintained on separate plates under conditions identical to those which are for the corresponding antiviral assays.

Daily aliquots of test compounds are made from stock solutions in individual tubes. On each day of treatment, daily aliquots of the diluted test compounds are suspended into culture medium at room temperature, and immediately added to the cell cultures, thereby subjecting each aliquot of test compound to the same, limited, number of freeze-thaw cycles.

HCV RNA levels are quantitatively measured using one of two methods. The first method used the application of commercial bDNA technology (Versant HCV™, Bayer Diagnostics, Inc., Oakland, Calif.) for the detection of intracellular HCV. For this assay, no RNA extraction is required. Cells are lysed in the culture wells, and the resulting solution is then directly quantitatively assayed for RNA. The bDNA assay uses the certified HCV international reference standards and has internal extraction controls included in each sample. The EC50 value for each test drug is calculated using linear regression analysis (MS Excel™).

The second method for HCV RNA quantitation is a modification of a previously described dot blot hybridization assay (Korba et al., Antiviral Res., 19(1):55-70 (1992)). Whole cell RNA is extracted from cells using either RNeasy™ mini-columns (Qiagen, Inc.), or Purescript RNA Purification kits (Gentra Systems, Inc.). RNA samples are denatured in 10×SSC/18% deionized formaldehyde for 20 min. at 80° C., are applied to nitrocellulose under vacuum, are washed once with 20×SSC, are baked for 15 min at 80° C. under vacuum, and are hybridized against ³²P-labelled DNA probes. Following the denaturation step, each RNA sample is split onto two nitrocellulose membranes for hybridization with either HCV-specific or human β-actin-specific ³²P-labelled DNA probes (95% of the sample for HCV, 5% for β-actin). The HCV hybridization probe can be a gel-purified, 6600 bp Hind III fragment which can be isolated from the HCV replicon source plasmid, BB7 (Blight et al., Science, 290:1972-1974 (2000)). The β-actin probe is a gel-purified, 550 bp PCR product generated from AVA5 cell RNA using a commercial PCR kit (Invitrogen, Inc.). Both probes are labeled with ³²P-dCTP using a commercial random priming procedure (Clonetech-BD Biosciences, Inc.). Hybridization is performed overnight at either 47° C. (HCV), or 40° C. (β-actin), and washing is performed at either 65° C. (HCV), or 60° C. (β-actin), as previously described (Korba et al., Antiviral Res., 19(1):55-70 (1992)). Quantitation against independently determined standards present on each hybridization membrane is achieved using a beta scanner (Packard Instruments, Inc.). The mean levels of β-actin RNA present in 6-8 untreated cultures contained in each experiment are used as the basis for determining the relative level of β-actin RNA in each individual sample. Levels of HCV RNA are normalized to the levels of β-actin RNA present in each individual sample. HCV RNA levels in treated cultures are then compared to the normalized mean levels of HCV RNA present in the 6-8 untreated cultures contained in each experiment. The EC50 value for each test drug is calculated using linear regression analysis (MS Excel™).

The Versant HCV™ method can be used to measure HCV viral load. Dot blot hybridization assay can be used to calculate the EC50.

### Example 5

### IL-29 in EAE Mouse Models for Multiple Sclerosis (MS)

Experimental allergic encephalomyelitis (EAE) is a mouse model for human MS (Gold et al., Mol. Med. Today, 6:88-91, 2000; Anderton et al., Immunol. Rev., 169:123-137, 1999). MS in humans can be broadly classified into chronic progressive and relapsing remitting disease phenotypes. These disease phenotypes can be modeled in mice using multiple methods. One such method of inducing chronic progressive MS in mice is to immunize mice with a peptide of the myelin protein MOG (myelin oligodendrocyte glycoprotein). This protein is present on the outside of the myelin sheath and acts as a protective layer for myelin. Mice are immunized sub-cutaneously with MOG peptide (MOG35-55) emulsified in RIBI adjuvant on day 0. Mice are then injected intravenously with pertussis toxin (PT) on day 2. The mice start showing symptoms of paralysis starting with a limp tail, wobbly motion, followed by hind limb and forelimb paralysis, which are scored according to several different parameters that measure the timing, extent and severity of disease. Delay in onset of disease indicates that the drug is modifying the disease process in mice. Decrease in incidence indicates that the drug is having an effect on the number of mice that are getting sick. Decrease in clinical score indicates that the drug has an effect on the severity of disease. Groups of mice are given PBS or either mouse IL28 or human IL-29. The onset of symptoms, incidence of disease scores and severity of disease scores in IL-28/29 treated mice indicates the effect of IL-28/29 on these parameters in this model. Mice (n=13/gp) are immunized s.c with 100 ug MOG35-55 in RIBI adjuvant on d0. All mice receive 200 ng pertussis toxin i.v on d2. Groups of mice are treated i.p with PBS, 25 ug human IL-29 every other day (EOD) on days 1-18 or with PBS, BSA or mouse IL28. As specified above, mice are scored for clinical signs and weight loss daily from d0-d30. IL-29 or mouse IL28 treated mice show a delay in the onset of disease compared to PBS treated animals.

To model relapsing remitting disease, mice (SJL) are immunized with a peptide derived from the proteolipid protein (PLP). Mice are immunized in the back s.c with PLP139-151 peptide emulsified in complete Freund's adjuvant (CFA). The mice start showing symptoms of paralysis starting with a limp tail, wobbly motion, followed by hind limb and forelimb paralysis, which are scored according to several different parameters that measure the timing, extent and severity of disease. In the RR-EAE model, during the course of disease, mice will spontaneously have a remission for a short period after which mice will again relapse spontaneously. Mice might have these relapsing-remitting cycles multiple times during the course of the disease. Delay in onset of disease indicates that the drug is modifying the disease process in mice. Decrease in incidence indicates that the drug is having an effect on the number of mice that are getting sick. Decrease in clinical score indicates that the drug has an effect on the severity of disease. In this model, decreases in numbers of relapses, decrease in maximum clinical score achieved at a relapse and to maintain mice in complete remission are all indications of a therapeutic drug. Groups of immunized mice are given either prophylactic (starting Day 3 after immunization) or therapeutic (starting on first day of clinical score) IL-29 or mouse IL-28 at different doses. Decrease in relapses, disease severity and incidence or induction of complete remission indicate that IL-29 or mouse IL-28 can inhibit RR-EAE and could be a therapeutic for RR-MS in humans.

### Prophylactic Administration of Mouse IL-28 or IL-29 Inhibits Severity of Disease and Disease Incidence in the RR-EAE Model in SJL Mice

Summary

To test if mouse IL-28 or human IL-29 had any effects on relapsing-remitting multiple sclerosis, the ability of mouse IL-28 or IL-29 to inhibit experimental autoimmune encephalomyelitis (EAE), a mouse model for RR-MS is tested. The well characterized proteolipid protein PLP 139-151 peptide immunization model in SJL mice is used. The experiment is run to determine that IL-28 or IL-29 could delay and/or inhibit disease scores in RR-EAE. Prophylactic administration of either IL-29 and mouse IL-28 can delay onset of disease and reduced incidence of disease in the RR-EAE model, suggesting that use of IL-28 or IL-29 may be beneficial in MS.

Study Design

Experimental autoimmune encephalomyelitis (EAE) is a mouse model for MS. In one such model, SJL mice are immunized with 100 μg PLP peptide (PLP139-151) emulsified in CFA adjuvant (1.1 ratio). A 1 mg/mL preparation of the PLP139-151 peptide in PBS is prepared. CFA (Sigma Aldrich Ltd) containing 1 mg/mL heat inactivated mycobacterium tuberculosis (Mtb) is fortified with further 1 mg/mL of Mtb (Difco Laboratories) to make a final concentration of 2 mg/mL Mtb. A 1:1 emulsion of this CFA and PLP peptide solution is generated using emulsifying syringes. The backs of mice are shaved and 100 μg PLP/CFA (200 uL of emulsion) is injected s.c in the backs of mice. Weights of mice are taken 2 days before and every day after the immunization. Mice are monitored daily for clinical scores. Groups of mice are injected i.p. with 100 μl PBS, or 5-75 ug mouse IL-28 or 25 ug IL-29 in a 100 uL volume EOD from days 3-23. In some experiments, 25 ug Novantrone was administered i.p. on Days 4, 8, 12 and 16. The weights of mice, clinical scores and incidence were evaluated and plotted for analysis.

### Therapeutic Administration of Mouse IL-28 or IL-29 Inhibits Severity of Disease and Relapse of Disease in the RR-EAE Model in SJL Mice

Summary

To test if therapeutic administration of mouse IL-28 or IL-29 would have any effects on relapsing-remitting multiple sclerosis, the ability of mouse IL-28 or IL-29 to inhibit experimental autoimmune encephalomyelitis (EAE), a mouse model for RR-MS is tested. The well characterized proteolipid protein PLP 139-151 peptide immunization model in SJL mice is used. The experiment is run to determine whether mouse IL-28 or IL-29 cand inhibit disease scores and prevent relapses when given therapeutically (after first clinical sign of disease) in RR-EAE.

Study Design

Experimental autoimmune encephalomyelitis (EAE) is a mouse model for MS. In one such model, SJL mice were immunized with 100 μg PLP peptide (PLP139-151) emulsified in CFA adjuvant (1.1 ratio). A 1 mg/mL preparation of the PLP139-151 peptide in PBS is prepared. CFA (Sigma Aldrich Ltd) containing 1 mg/mL heat inactivated mycobacterium tuberculosis (Mtb) is fortified with further 1 mg/mL of Mtb (Difco Laboratories) to make a final concentration of 2 mg/mL Mtb. A 1:1 emulsion of this CFA and PLP peptide solution is generated using emulsifying syringes. The backs of mice are shaved and 100 μg PLP/CFA (200 uL of emulsion) is injected s.c in the backs of mice. Weights of mice are taken 2 days before and every day after the immunization. Mice are monitored daily for clinical scores. Groups of mice are injected i.p. with 100 μl PBS, or 25 ug or 50 ug mouse IL-28 or human IL-29 in a 100 uL volume every day (ED) for 30 days starting from Day 1 of first clinical score in each individual mice. The weights of mice, clinical scores and incidence are evaluated and plotted for analysis.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. An isolated polypeptide consisting of amino acid residues 1-161 of SEQ ID NO:6.

2.-7. (canceled)

8. The polypeptide claim 1, wherein the polypeptide further comprises a polyethylene glycol moiety covalently attached to the N-terminus of the polypeptide.

9. The polypeptide of claim 8, wherein the polyethylene glycol moiety is mPEG propionaldehyde.

10. The polypeptide of claim 8, wherein the polyethylene glycol moiety has a molecular weight of about 20 kD, 30 kD or 40 kD.

11. The polypeptide of claim 8, wherein the polyethylene glycol moiety is linear.

12. (canceled)

13. An isolated polynucleotide encoding the polypeptide of claim 1.

14. The isolated polynucleotide of claim 13, wherein the polynucleotide comprises the nucleotides of SEQ ID NO:5.

15. An expression vector comprising the following operably linked elements:
a transcription promoter;
a DNA segment encoding the polypeptide of claim 1; and
a transcription terminator.

16. A cultured cell comprising the expression vector of claim 15, wherein the cell expresses the polypeptide encoded by the DNA segment.

17. A method of producing a polypeptide comprising:
culturing a cell comprising the expression vector of claim 15, wherein the cell expresses the polypeptide encoded by the DNA segment; and
recovering the expressed polypeptide.

18. An antibody or antibody fragment that specifically binds to the polypeptide of claim 1.

19. The antibody or antibody fragment of claim 18, wherein the antibody is selected from the group consisting of a polyclonal antibody, a murine monoclonal antibody, a humanized antibody derived from a murine monoclonal antibody, an antibody fragment, neutralizing antibody, and a human monoclonal antibody.

20. The antibody fragment of claim 18, wherein the antibody fragment is selected from the group consisting of F(ab′), F(ab), Fab′, Fab, Fv, scFv, and minimal recognition unit.

21. A composition comprising:
an isolated polypeptide according to claim 1; and
a pharmaceutically acceptable vehicle.

22. (canceled)

23. A method of treating a patient having a hepatitis B or hepatitis C infection comprising administering to the patient a therapeutically effective amount of the composition of claim 21, wherein after administration of the composition the viral load is reduced or viral replication is inhibited.

24.-27. (canceled)
